(19) United States
(12) Patent Application Publication
(10) Pub. No.: US 2016/0299129 A1
(43) Pub. Date: Oct. 13, 2016

(54) Ultra Sensitive and Specific Multiplex Biosensor System Based on Multiple Cooperative Interactions

(71) Applicant: Xiaolei Qiu, San Ramon, CA (US)

(72) Inventor: Xiaolei Qiu, San Ramon, CA (US)

(21) Appl. No.: 15/092,899

(22) Filed: Apr. 7, 2016

Related U.S. Application Data
(60) Provisional application No. 62/143,860, filed on Apr. 7, 2015.

(51) Int. Cl. G01N 33/53; C12Q 1/70

(52) U.S. Cl. CPC G01N 33/5306; G01N 33/5308; C12Q 1/708

(57) ABSTRACT

Methods for implementing a biosensor system for highly sensitive and highly specific detection of a biological analyte are disclosed. The detecting methods include interacting the analyte to at least two or more sensitive elements (SE) and two or more signal transducing/amplifying molecules (TA) that form a stable sensitive-element-signal transducer/amplifier complex (SETAC) for further detection. Sequential format and concurrent format of the methods are disclosed. Multiplex format and automated format of the methods are disclosed. Biosensor systems and assay kit implementing these methods are disclosed.

### CROSS-REFERENCE TO RELATED APPLICATION(S)

This application claims the benefit of U.S. provisional patent application Ser. No. 62/143,860, filed Apr. 7, 2015 and entitled “An Ultra Sensitive and Specific Multiplex Biosensor System Based on Multiple Cooperative Interactions”. The aforementioned U.S. provisional patent application is herein expressly incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF INVENTION

The present invention is in the field of biological analyte detection. The invention includes methods for implementing a biosensor system directed to the detection of nucleic acids, proteins, epigenetic markers, modifications of biological molecules, or cellular components in a biological sample, methods for using at least two sensitive elements and at least two primary signal amplifying molecules that form a stable complex that enable the specific detection of the analyte in a biological sample. The invention further includes various formats of the methods, and the system and the kit that are related to the method. It also includes a method that specifically used for the detection of HPV nucleic acids. New techniques in various embodiments are provided for substantially increasing analyte specific signal, eliminating nonspecific binding, therefore reducing the background noise resulting in an increased the signal-to-noise ratio.

### BACKGROUND

A biosensor system is a physical biological device that detects and measures specific analytes. A typical biosensor system consists of 1) one or more biological Sensitive Elements (SE) that specifically interact with the analyte. High specificity of the SE to the analyte of interest among a matrix of other components in the biological sample is a critical requirement. 2) one or more signal Transducers/Amplifiers (TA) that transform and amplify the signal from the interaction between the SE and the analyte into a measurable and quantifiable signal. Various types of biosensors are commonly used and are described below.

Nucleic acid detection is one of the most common applications in a biosensor system. It may also be referred to as genosensor. This type of biosensor often is used to detect, in a biological sample, nucleic acids with a known sequence. When the target nucleic acid sequence is known, the complementary sequences may be designed, synthesized and used as the SE. When interacting with the sample, these sensitive elements recognize, interact and hybridize to the particular regions of the nucleic acid. A detection step is further implemented for detecting and quantifying the target.

Antibody-antigen interactions may also be used in a biosensor, in particular an immuno-sensor. An antibody may be used as the SE to detect a particular antigen in a biological sample. One or more antibodies may have very specific binding affinity for a specific antigen.

The catalytic activity of enzymes may also be used in a biosensor. Enzymes may be used as the SE to catalyze analyte specific reaction. Enzymes may convert, inhibit or activate the analyte to a detectable form.

Many methods have been invented to improve the specificity, i.e. higher signal-to-noise (S/N) ratio, of biosensor systems. One example is the “sandwich” assay system, which utilizes a first SE to capture the analyte followed by a washing step to remove most non-specific targets, and then detects the analyte with a second SE that interacts with the analyte through a different region or domain (Lequin R M et al 2005; Urdea M S et al. 1993). While this approach works well in a system where most of the non-specific targets may be removed after the analyte is captured, it may not be applied to systems such as intact tissues or cells, where all the potential non-specific targets for either the first and/or the second SE stay within the system throughout the assay procedure.

Another example is the conventional assay system that uses one single SE to recognize an analyte. In order to ensure that all the analytes are being detected, excessive amount of SE is normally added, and followed by a washing step to remove the un-reacted SE, One or more TA are used in excess to amplify the signal for detection. Washing steps are required between any two steps to remove the excessive material before the next material may be added. This type of assay contains specific incubation condition to allow the interactions of analyte-SE and SE-TA to be strong and stable. The washing buffer is also formulated to wash away the excessive materials, including SE and TA that are non-specifically bound to non-analyte targets. Such non-specific binding may not be removed during washing if the binding strength is above certain level. Therefore, it is difficult to distinguish analytes with medium to high level of homology. The residual non-specific binding is the cause of the high background or false-positive signal.

Therefore, major challenges with a biosensor system remain to be 1) to have sufficient signal amplification 2) to ensure that only the specific interaction between the SE and the analyte is amplified, while interactions between the SE and other non-specific targets are not detected, and 3) to minimize background signal generated by the TA itself. There exists a need for methods, systems, and kit product for implementing a novel biosensor to address these challenges and shortcoming of conventional approaches.

### SUMMARY

Disclosed are methods, systems, and kit products for implementing biosensor systems for the detection of an analyte in a biological system with enhanced signal, reduced noise, and increased specificity in various embodiments. Some first embodiments are directed at a method for implementing biosensor systems. In these embodiments, an analyte of interest may be recognized, interacted and bound to at least two sensitive elements (SE). Each sensitive element also binds to at least one specific signal transducer/amplifier. Therefore, two or more signal transducer/amplifier molecule (TA) are used for each to specifically recognize one of the SE or the other TA and bind to them. The TA molecules are further interacted with or bound to a labeling/detecting system, which generates signal that may be detected and quantified in various ways.

Any of the reactions in various embodiments occur under a designed incubation condition, in which conditions such as the pH of the buffer, the salt concentration of the buffer, the incubation temperature, and the incubation time may be adjusted. These conditions are designed to provide a stringency allowing the bonding between the two species to form at different strength or stability. This stringency-dependent interaction may be substantially strong or stable, such that the complex containing the bonded components remains intact during incubation as well as the subsequent washing procedures. The interaction may be weak or sub-stable, such that the bonding between interacting components does not form efficiently during incubation and may be interrupted or broken during the subsequent washing steps.

In some embodiments, under a designed incubation condition, the two or more sensitive elements (SE) and the two or more signal transducer/amplifier (TA) are bonded to form a stable sensitive-element-signal transducer/amplifier complex (SETAC) that binds to the analyte.

In some embodiments, upon the formation of the SETAC that binds to the analyte, signal is generated, detected, and quantified by the labeling/detection system to reflect the amount of the analyte present in the biological sample.

In some embodiments, there may be additionally one or more layers of the secondary signal amplifier molecules (SA) that bind to the SETAC. Each higher order layer of the SA interacts specifically with the immediate previous layer under a designed reaction/incubation condition to form a stringency-dependent interaction. The purpose of using one or more layers of the secondary signal amplifiers is to further amplify the signal enabling the possibility of detecting and quantifying low amount of analyte in a biological sample. Upon formation of the complex composed of the SETAC and secondary amplifiers, signal is generate, detected, and quantified by the labeling/detection system to reflect the amount of the analyte present in the biological sample.

In some embodiments, the analyte may be a nucleic acid, an antigen, a protein or peptide, or an antibody. If it is a nucleic acid to be detected, the SE are probes, which are nucleic acids and nucleic acid derivatives that are complementary to the nucleic acid target of interest. If it is an antigen to be detected, the SE may be monoclonal antibodies, polyclonal antibodies, or fragments of antibodies that specifically bind to the antigen of interest. If it is an antibody to be detected, the SE may be peptides, peptide fragments or fragments of naturally occurred proteins that may specifically recognized by the antibody of interest.

The biological samples may be a cultured cell, a cell separated from a tissue, a cell from a clinical sample, a tissue, a fresh-frozen tissue, a fixed tissue, a fixed and embedded tissue, such as formalin fixed and paraffin embedded (FFPE) tissue, blood, serum, or any body fluid. The biological samples may not need further process; therefore the consequence detection of the analyte occurs in situ. The biological samples may need further process to expose the analyte of interest for the SE to bind to. The analyte may be immobilized on a solid medium such as membranes, glass or plastic slides, beads, test strips, or multi-well plates.

In some embodiments, the TA may be linked to a detecting system that contains labeling molecules. The label molecules may be directly or in-directly linked to the TA. The direct label molecules may be a fluorescent label or a radioactive label, with which the label itself generates quantifiable signal intensity. The indirect label molecules may be an enzyme label, with which a further step is needed for an enzymatic reaction to occur and to produce another product that generates quantifiable signal intensity. For example, a horse radish peroxidase (HRP) enzyme system may be used with a substrate such as TMB to produce chromogenic product for optical density to be measured in solution. It may be used with another substrate such as DAB to produce colored precipitation for the staining intensity to be examined under a microscope. Some more examples of different enzyme systems including alkaline phosphatase (AP), tyramide signal amplification system, and chemiluminescent system, such as luciferase/luciferin system.

Some of the embodiments are directed to a system that contains at least two SETAC that recognize the same analyte. The purpose of this design is to further improve the detection sensitivity and signal-to-noise ratio.

Some of the embodiments are directed to a biosensor method for detecting a nucleic acid, an antigen or an antibody, which contains sequential steps such that the interaction of the analyate and capturing molecule, the interaction of the analyte and SE, the interaction of TA and SE, the interaction of secondary amplifiers (SA) and TA, and the interaction of the label molecules and the TA or SA are individual assay steps. Optionally washing steps after each incubation step are carried out to remove the excessive amount of the reagents and the unbound species.

Some of the embodiments are directed to a biosensor method for detecting a nucleic acid, an antigen or an antibody, which contains concurrent steps such that the two or more TA may be combined and incubated concurrently, followed by an optional washing step to remove the excessive amount of the reagents and the unbound species.

Some of the embodiments are directed to a system that allows the detection of two or more analytes simultaneously. The system contains two or more sets of SETAC that interact with distinct analytes, distinct optional secondary amplifiers, and distinct labeling/detection system, such that two or more analytes generate distinct signals that may be detected and quantified differentially.

Some of the embodiments are directed to a reagent kit that contains at least two sensitive elements, at least two signal transducing/amplifying molecules, and a detection system/molecule.

Some of the embodiments are directed to a method for implementing a biosensor system for detection and quantification of human papillomavirus nucleic acids in a sample. The method includes the process for obtaining a biological sample that contains cells infected by HPV. The cells in the sample are to interact with at least one pair of SE that specifically recognizes and binds to the HPV16 or HPV 18 nucleic acids in the cells. Two TA are subsequently reacted with the HPV16 or HPV18 probes to form a stable SETAC complex. HRP enzyme system is labeled on the signal amplifying probe. A tyramide based signal amplification is used, followed by DAB substrate reaction to produce brown color precipitation. The darkness of the color represents the quantification of the HPV16/18 nucleic acids present in the cell sample.

### BRIEF DESCRIPTION OF THE DRAWINGS

The drawings illustrate the design and utility of various embodiments of the invention. It should be noted that the figures are not drawn to scale and that elements of similar structures or functions are represented by like reference numerals throughout the figures. In order to better appreciate how to obtain the above-recited and other advantages and objects of various embodiments of the invention, a more detailed description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 illustrates a schematic diagram for implementing the biosensor system in one or more embodiments that comprises one stable Sensitive Element and Transducer/Amplifier Complex (SETAC), which comprises at least two sensitive elements (SE) interacting with the analyte and two or more signal transducer/amplifiers (TA) interacting with either the SE or the other TA.

FIG. 2 illustrates a schematic diagram for implementing the biosensor system in one or more embodiments that comprise one stable SETAC as illustrated in FIG. 1 that further comprises all the interactions between SE and TA are sub-stable, and one or more TA also interact another TA through sub-stable interaction, under designed assay conditions.

FIG. 3 illustrates a schematic diagram for implementing the biosensor system in one or more embodiments that comprise one stable SETAC as illustrated in FIG. 1 that further comprises all the interactions between SE and TA, and between TA and TA are sub-stable, under designed assay conditions.

FIG. 4 illustrates a schematic diagram for implementing the biosensor system in one or more embodiments that comprise one stable SETAC in which the SE also interact with other SE through sub-stable interaction.

FIG. 5 illustrates a schematic diagram for implementing the biosensor system in one or more embodiments that comprise one stable SETAC as illustrated in FIG. 1, FIG. 2, FIG. 3, and FIG. 4 that further comprises labeling molecules or particles directly associate with the SETAC for signal detection.

FIG. 6 illustrates a schematic diagram for implementing the biosensor system in one or more embodiments that comprise one stable SETAC as illustrated in FIG. 1, FIG. 2, FIG. 3, and FIG. 4 that further comprises one or more layers of secondary amplifiers (SA) for further signal amplification.

FIG. 7 illustrates a schematic diagram for implementing the biosensor system in one or more embodiments that the secondary amplifiers (SA) may also interact with TA through sub-stable interaction.

FIG. 8 illustrates a schematic diagram for implementing the biosensor system in one or more embodiments as illustrated in FIG. 5, FIG. 6 and FIG. 7 that further comprises labeling molecules or particles for indirect signal detection.

FIG. 9A illustrates a schematic diagram for implementing the biosensor system in one or more embodiments that comprises two or more SETAC for the same analyate.

FIG. 9B illustrates a schematic diagram for the multiple SETAC for the same analyte shown in FIG. 9A, in which the SE in each SETAC are different while the same TA are used for all the SETAC.

FIG. 10 illustrates a schematic diagram for implementing the biosensor system in one or more embodiments that comprise two or more SETAC for the same analyte as illustrated in FIG. 9, that further comprises one or more layers of SA for further signal amplification.

FIG. 11A illustrates a schematic diagram for implementing the biosensor system in one or more embodiments that comprise two or more distinct sets of SETAC for two or more distinct analytes. SE in each SETAC are different, while the same set of TA is used for all the SETAC for the same analyte. Distinct analyte has distinct set of TA.

FIG. 11B illustrates a schematic diagram for implementing the biosensor system in one or more embodiments that comprise two or more distinct analytes to be detected simultaneously with distinct sets of amplifier and label molecules.

FIG. 12 illustrates a schematic diagram for implementing the biosensor system in one or more embodiments that comprises a solid media for the analyte to be immobilized to.

FIG. 13 illustrates a schematic diagram for implementing the biosensor system in one or more embodiments that the analyte may be detected directly in a biological specimen.

FIG. 14 illustrates a schematic diagram for implementing the biosensor system in one or more embodiments that comprises two or more SE/probes interacting with the nucleic acid analyte, two or more TA, one or more additional layers of secondary amplifier (SA), and direct or indirect labeling molecules or particles.

FIG. 15 illustrates a schematic diagram for implementing the biosensor system in one or more embodiments, in which the orientation and configuration of the SE/probes and the TA may be different.

FIG. 16 illustrates a schematic diagram for implementing the biosensor system in one or more embodiments, in which two or more SE/probes in an SE/probe-TA complex (SETAC) interact with the nucleic acid analyte through sub-stable interactions and also interact with other SE/probes through sub-stable interaction.

FIG. 17 illustrates a schematic diagram for implementing the biosensor system in one or more embodiments that comprise two or more SE/probe-TA complexes (SETAC), as illustrated in FIG. 9 and FIG. 10, which comprises different SE/probes, but same set of TA.

FIG. 18 illustrates a schematic diagram for implementing the biosensor system in one or more embodiments, in which one or more layers of SA interact with TA, as illustrated in FIG. 7, through sub-stable interaction for detecting nucleic acid analytes.

FIG. 19 illustrates a schematic diagram for implementing the biosensor system in one or more embodiments that comprises two or more SE that each comprises an antibody part that interacts with the analyte, and a nucleic acid part that interact with TA through sub-stable interaction.

FIG. 20 illustrates in-situ hybridization images using CASKI, HELA and C33A cell lines to represent the biological sample in one or more embodiments, in which both SE and TA have the nature of nucleic acid. Two sensitive element sets (HPV 16 and HPV 18 target probes respectively), and a set of two TA (in this case, primary signal amplifying probes) were used.

FIG. 21A illustrates in-situ hybridization images using CASKI, HELA and C33A cultured cells to represent the biological sample in one or more embodiments, in which only one of the probes (half set of each SE) in HPV 16 target probe pairs were used.

FIG. 21B illustrates in-situ hybridization images using CASKI, HELA and C33A cultured cells to represent the biological sample in one or more embodiments, in which only one of the probes (half set of each SE) in HPV 18 target probe pairs was used.

FIG. 22A illustrates in-situ hybridization images using CASKI, HELA and C33A cultured cells to represent the biological sample in one or more embodiments, in which both HPV 16 probe sets (full SE set) but only one of the two TA was used.

FIG. 22B illustrates in-situ hybridization images using CASKI, HELA and C33A cultured cells to represent the biological sample in one or more embodiments, in which both HPV 18 probe sets (full SE set) but only one of the two TA was used.

### DETAILED DESCRIPTION

Various embodiments are directed to a method, system, composition and kit for the detection of biological analyte, such as, nucleic acid, protein, peptide, or antigen, in a biological sample through multiple cooperative interactions. Other objects, features, and advantages of the invention are described in the detailed description, figures, and claims.

In various embodiments, a biosensor system is introduced. Such system is a physical biological system that detects and measures specific analyte. Such system comprises (1) a target specific element that may specifically recognize and interact with the target/analyte of interest in a biological sample, (2) an amplifying element that functions as a linker between the target specific element and the detection element while providing multiple binding sites for the detection element and consequently amplifies the signal, and (3) a detection element that may specifically recognize and interact with the signal amplifying element, and through various mechanism it generates signals for detection and quantification.

The target specific element may be referred to a biological Sensitive Element (SE).

The amplifying element may be referred to a signal Transducer/Amplifier (TA) that transforms, and amplifies the signal from the interaction between the SE and the analyte of interest into a measurable and quantifiable signal for detection.

The detection element may be referred to a labeling molecule or particle that specifically binds to the signal amplifier for signal detection and quantification.

Each of the binding events occur under a designed assay condition, in which the pH of the incubation buffer, the salt concentration of the incubation buffer, the incubation temperature, the incubation time, as well as the composition of the wash buffer, the washing temperature, and the washing time, may be adjusted in order to control the strength of the binding. The assay conditions create a stringency-dependent environment allowing bindings with different strength to occur. A strong interaction may be desired in order to form a stable interaction to minimize the separation of the components during incubation and upon washing. A sub-stable interaction may be desired in order to form a sub-stable interaction to minimize non-specific binding. A component that interacts with two or more other components through sub-stable interaction may be held stably through the cooperative sub-stable interactions to its interacting partners.

When all the SE and the TA are present and interacting with their interacting partners through stable or sub-stable interactions, they form a stable Sensitive Element and Transducer/Amplifier Complex (SETAC), under the designed assay conditions.

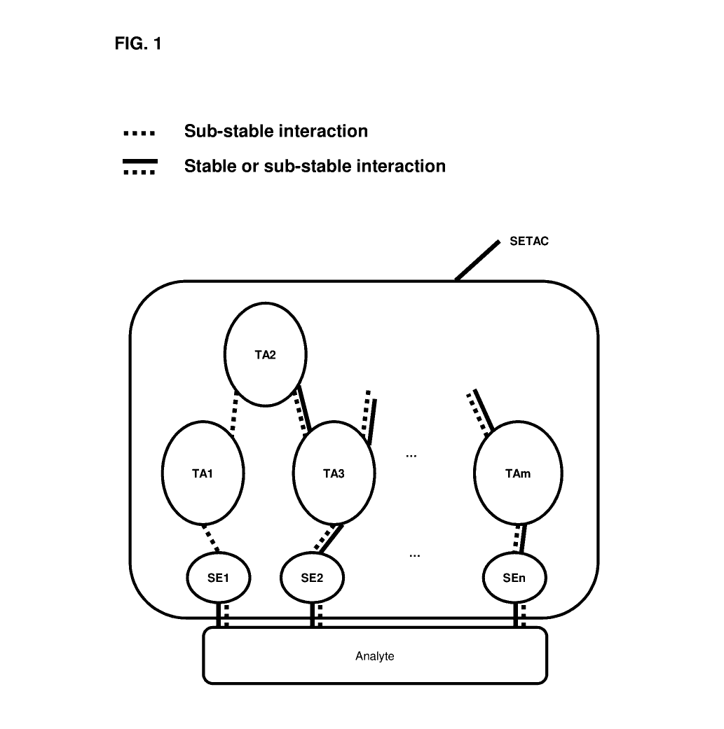

Various embodiments of the methods, systems, and kits will now be described in detail with reference to the drawings, which are provided as illustrative examples of the invention so as to enable those skilled in the art to practice the invention. Notably, the figures and the examples below are not meant to limit the scope of various embodiments, unless otherwise specifically described in particular embodiment(s) or recited in the claim(s).

Where certain elements of embodiments may be partially or fully implemented using known components, methods or processes, portions of such known components, methods or processes that are necessary for an understanding of the present invention will be described, and the detailed descriptions of other portions of such known components, methods or processes will be omitted for ease of explanation and to not obscure embodiments of the invention. Further, embodiments encompass present and future known equivalents to the components referred to herein by way of illustration. More details about various processes to implement various embodiments are further described below with reference to FIGS. 1-21.

FIG. 1 illustrates a schematic diagram for implementing a biosensor system in one or more embodiments that comprises a stable Sensitive Element and Transducer/Amplifier Complex (SETAC). In some embodiments, two or more SE are required (denoted as SE1, SE2 . . . SEn), and two or more TA are required (denoted as TA1, TA2 . . . TAm). Under a designed assay condition, the two or more TA interact with the SE or other TA, and there is no interaction among the SE. All the SE may bind to the analyte through a stable or a sub-stable interaction. Each SE interacts specifically with a TA. At least one SE interacts with a TA through sub-stable interaction, and at least one TA interacts another TA through sub-stable interaction. Only when all the SE and TA are present and interacting with their interacting partner, do they form a stable SETAC structure through multiple cooperative interactions. If one or more components in this complex are missing, the structure falls apart under the designed assay conditions or during subsequent washing step.

The benefit of such design is that if an SE binds non-specifically to an off-target, a stable SETAC may not form without the presence of all the other SE. Because the chance that all the SE bind to the same non-specific substance within close proximity is very low, this design will result in further improved signal-to-noise ratio comparing to using a single SE to recognize the analyte. Furthermore, because the detectable signal is generated through TA, within an SETAC, each TA is capable of generating signal, and the collective signal generated by the SETAC is the sum of the signal generated by all the composing TA. Therefore, when at least two TA are present in the SETAC, the collective SETAC signal is at least 2 times as high as the signal generated by a single TA. In addition, because the integrity of the SETAC requires the presence of all the SE and TA, if a TA non-specifically binds to a non-specific substance, the SETAC will not assemble upon it because of the absence of the other SETAC components (SE and TA). Therefore, even though a non-specifically bound TA may be capable of generating background signal, the level of signal is only a portion of that generated by the full SETAC that associates with the analyte, leading to improved signal-to-noise ratio.

The analyte may refer to a target of interest in a biological sample. Examples of the target include nucleic acid, protein, peptide, or antigen. Examples of the nucleic acid as the analyte include DNA, messenger RNA (mRNA), and non-messenger RNA that is selected from the group consisting of transfer RNAs (tRNA), ribosomal RNA (rRNA), snoRNA, microRNA, siRNA, snRNA, exRNA, piRNA and scaRNA. Examples of the biological sample include fresh cells, fixed cells, fresh tissue, fresh frozen tissue, formalin-fixed and paraffin-embedded (FFPE) tissue, blood, serum, or any body fluid.

The term “incubation” refers to the process that allows at least a portion of the two elements interact with each other, at certain condition, bind to each other to form a complex.

The term “hybridization” refers to the process that when at least a portion of the two nucleic acid molecules are complementary to each other, at certain condition, the complementary regions of the two molecules will anneal, or bind to each other to form a stable duplex. This may be referred to a stable interaction. Upon the change of the temperature, at least a portion of the duplex will dissociate and become two separate single stranded nucleic acid molecules. This temperature-dependent equilibrium process between the annealed state and the separate state is a dynamic process:

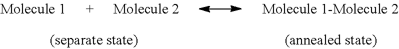

Tₘ, the melting temperature, is defined in the field to describe this dynamic equilibrium. At this temperature, there is 50% of the nucleic acid molecules existing as single strands, and the other 50% existing as duplex. The state when more than or equal to 50% of the molecules exists as duplex is a stable state that is resulted from a stable interaction. The state when less than 50% of the molecules exist as separate molecules is a sub-stable state that is resulted from a sub-stable interaction.

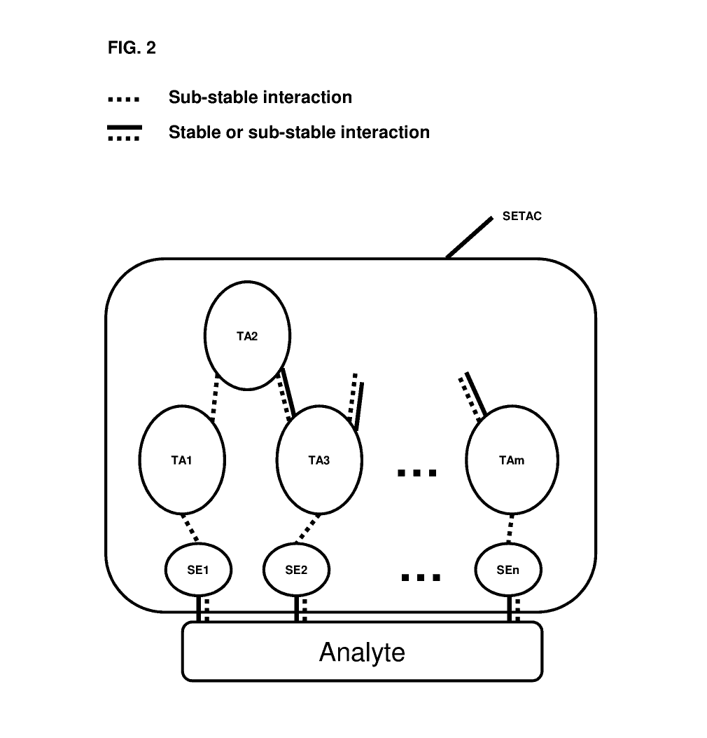

In some embodiments, as shown in FIG. 2, all the SE within the SETAC interact with TA through a sub-stable interaction, such that any non-specifically bound SE may not generate significant background signal because its interaction with the TA is sub-stable and not strong enough to sustain a SETAC.

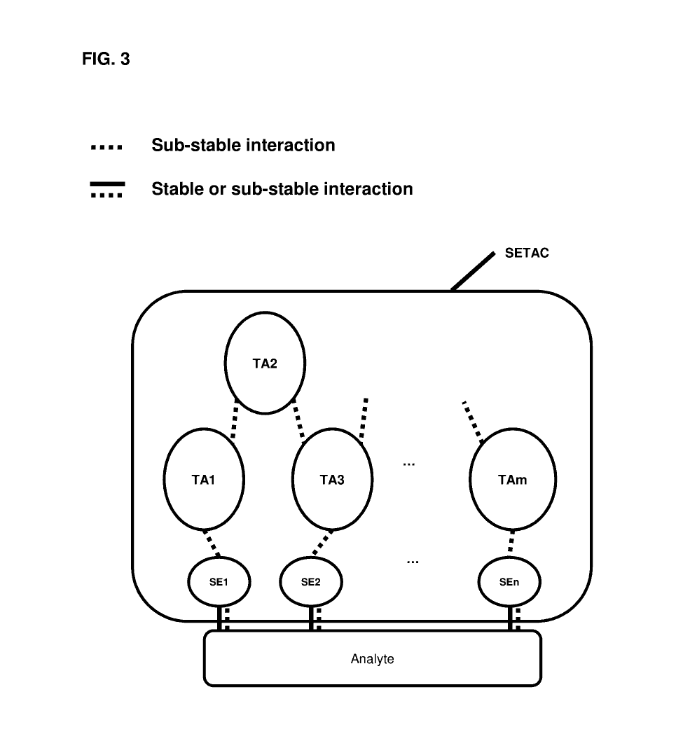

In some embodiments, as shown in FIG. 3, all the SE within the SETAC interact with TA through a sub-stable interaction, and all the TA interact with other TA through sub-stable interactions. With this arrangement, none of the non-specifically bound SE will generate significant background signal. Furthermore, any non-specifically bound TA will generate substantially lower background signal than the analyte may generate, because an analyte may have multiple SETAC, and the signal generated from a single TA is only a portion of the signal generated by an SETAC which contains multiple TA. This results in further improvement in signal-to-noise ratio.

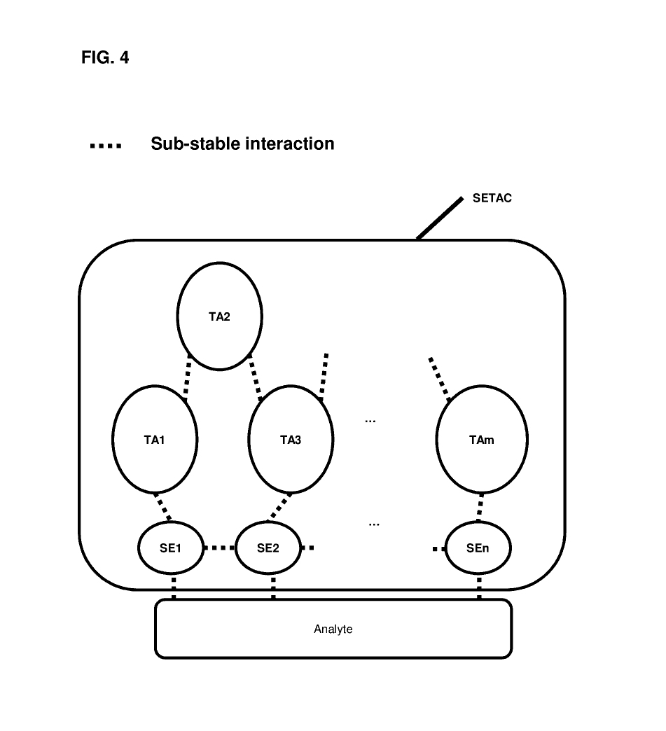

In some embodiments, as shown in FIG. 4, one or more SE interact with the analyte through sub-stable interaction and also interact with another SE through sub-stable interaction. The benefit of this design is that if the SE/analyte interaction is weak, the interaction may be enhanced by the sub-stable interaction with another SE that also binds specifically to the analyte, thus results in both increased sensitivity, as well as specificity.

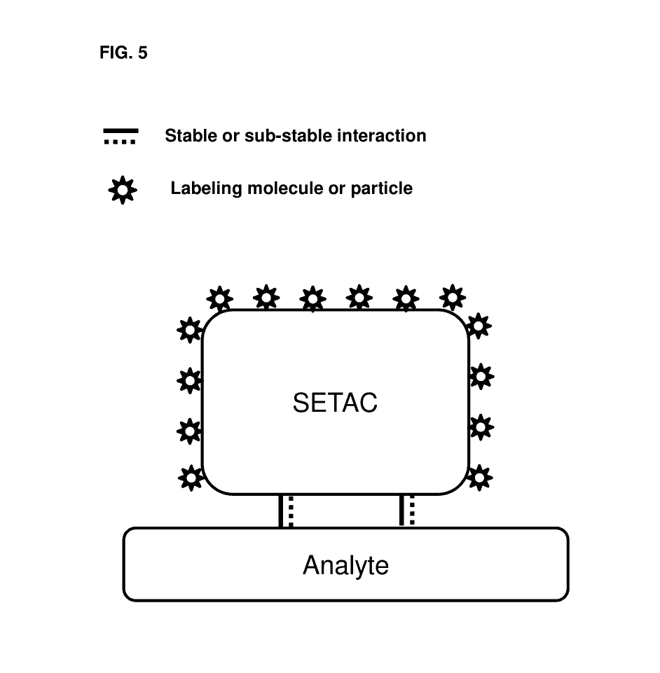

In some embodiments, as shown in FIG. 5, the labeling molecules or particles may bind to or directly conjugated to the TA in the SETAC.

The labeling molecules or particles are materials that generate detectable signal through various mechanism comprising direct labeling and indirect labeling. Direct labeling refers to the mechanisms that the signal is detected directly on the labeling molecules or particles without the need to use other materials such as a substrate. Direct labeling comprises fluorescent labeling, colormetric labeling, radioactive labeling, isotopic elements, and barcode labeling.

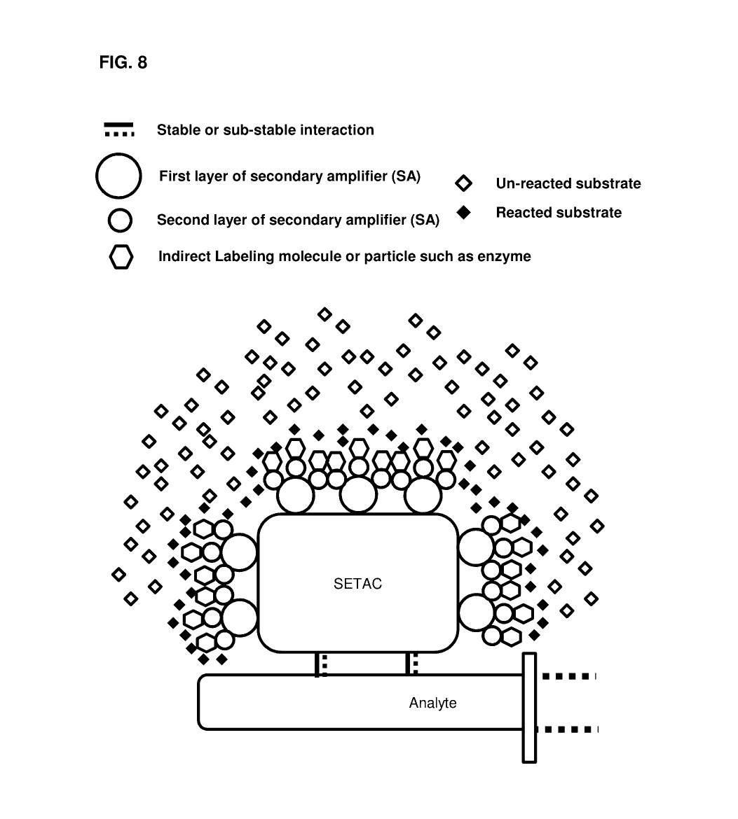

Indirect labeling refers to the mechanisms that additional reaction with the labeling molecules or particles is required in order for the signal to be generated and detected. For example, as shown in FIG. 8, an enzyme-substrate reaction. Indirect labeling comprises colorimetric detection, chromogen precipitation, tyramide signal amplification system, and chemiluminescent detection. Multiple indirect signal generating steps may be used sequentially to further amplify the signal. Substrate is required in order for the indirect labeling molecules or particles to be detected.

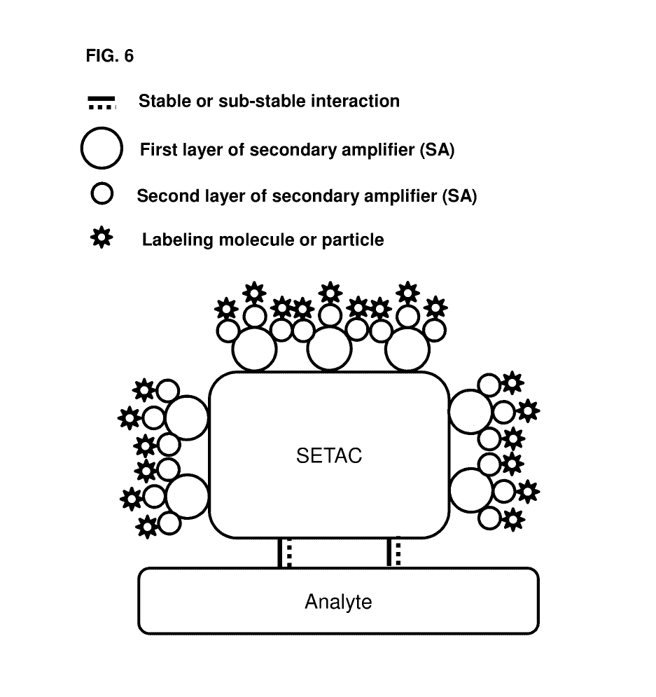

In some other embodiment, as shown in FIG. 6, one or more layers of SA may be used for signal amplification. The SA may be chosen such that there are multiple molecules of the first layer of the SAbind to each TA, multiple molecules of the second layer of the SA bind to the first layer of the SA, so on and so forth. This binding arrangement allows multiplied detection molecules to be bound; therefore the signal is exponentially amplified.

Examples of the SA include a nucleic acid, an oligonucleotide, a deoxynucleotide, a ribonucleotide, a nucleotide derivative, a monoclonal antibody, a fragment of monoclonal antibody, a polyclonal antibody, a fragment of polyclonal antibody, a protein, a fragment of protein, a peptide, a fragment of peptide, an antigen, a hapten, a biotin, an avidin, a streptavidin, a tyramide signal amplification system, an enzymes, a particle, and or or a mixture of what is listed above.

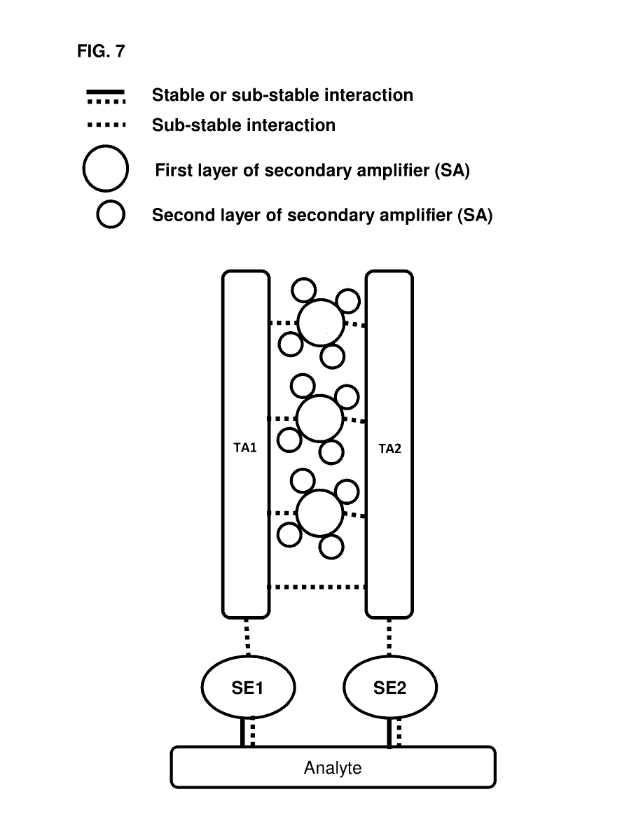

In one or more embodiments, as shown in FIG. 7, the multiple SA bind to two or more TA through sub-stable interaction, such that only if all the TA are present, may the SA stably bind to the TA through cooperative sub-stable interaction. The benefit of this design is that a non-specifically bound TA cannot recruit the SA without the presence of other TA that the SA bind to, thus will not generate detectable background signal, which results in further improved signal-to-noise ratio.

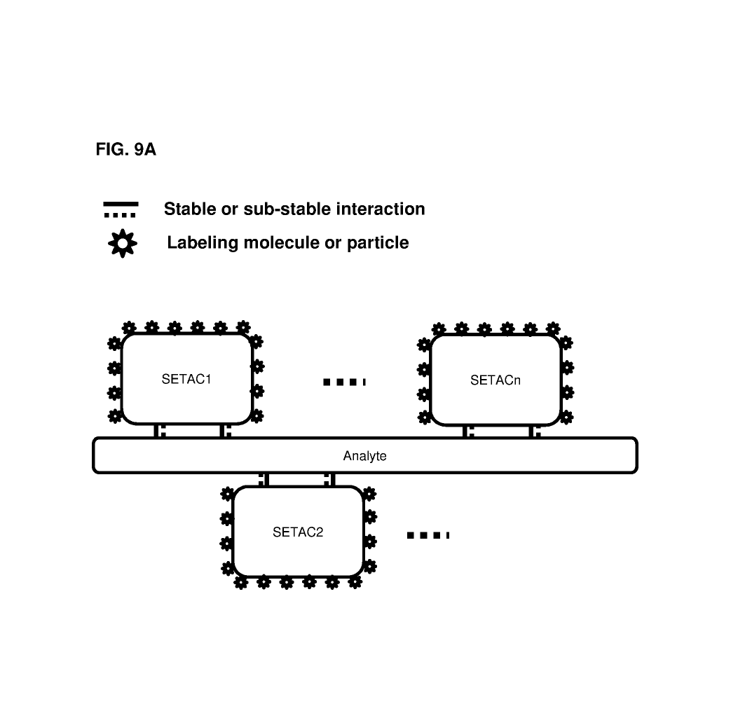

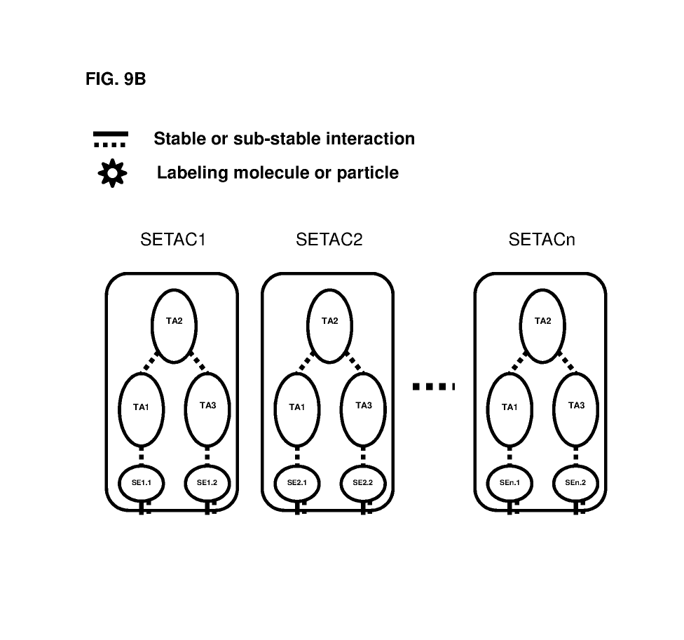

In one or more embodiments, as shown in FIG. 9, the analyte binds to two or more SETAC. For each analyte, the TA elements from one SETAC are identical to the TA elements in another SETAC, whereas the SE elements in each SETAC bind to different regions of the analyte. This design results in multiplication of signal generated from a single SETAC, leading to an increased sensitivity. Because the background signal generated by TA remains the same, this design also results in further improved signal-to-noise ratio.

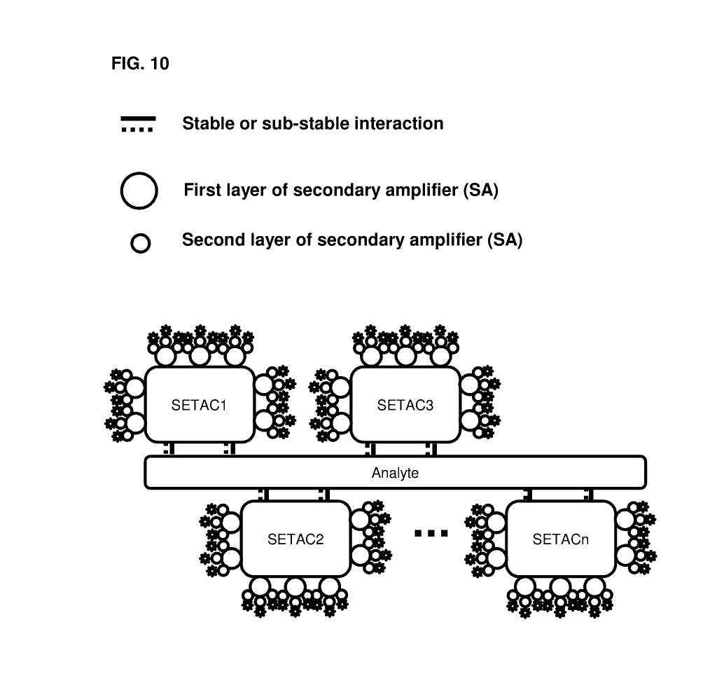

In one or more embodiments, as shown in FIG. 10, the analyte binds to two or more SETAC in a manner similar to that shown in FIG. 9. One or more layers of SA is used to further amplify signal, in a similar manner as shown in FIG. 6.

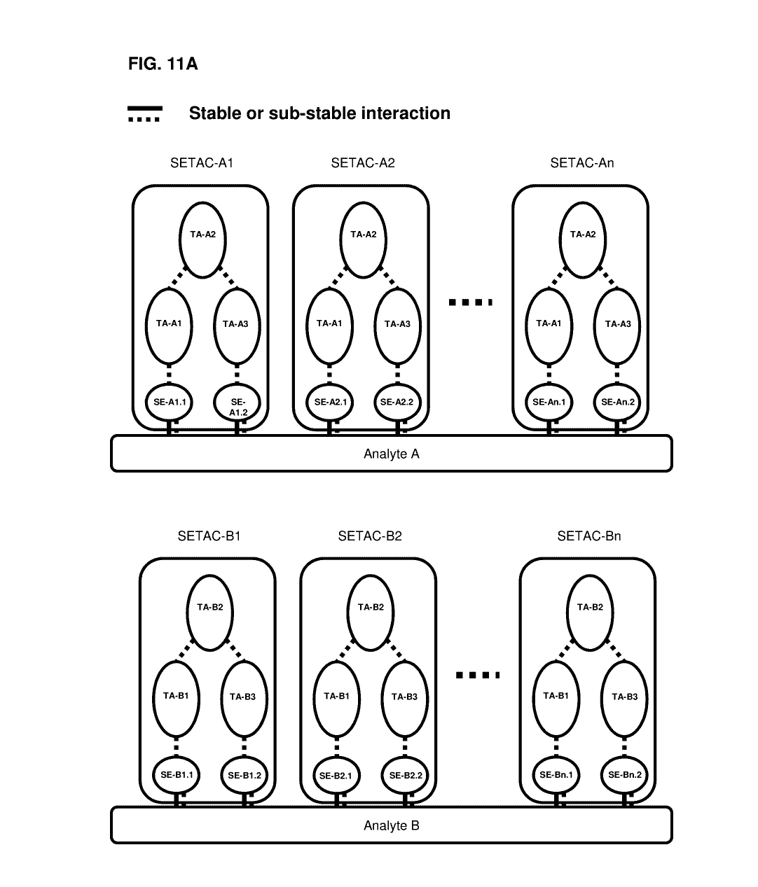

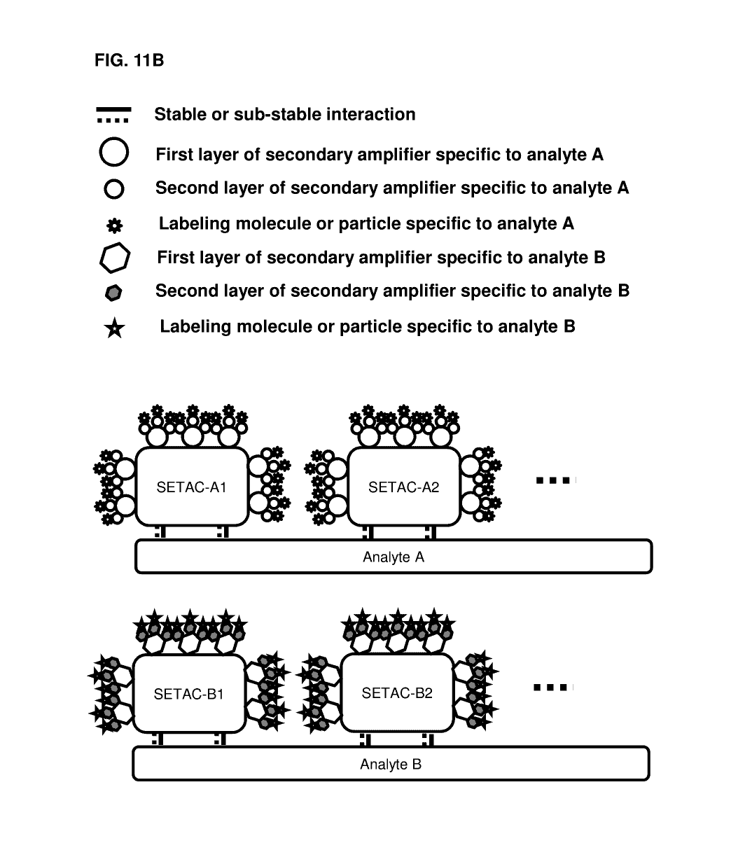

In one or more embodiments, as shown in FIG. 11, two or more analytes are detected simultaneously in a multiplex format. As shown in FIG. 11A, to detect analyte A and analyte B simultaneously, a first series of SETAC (SETAC-A1, SETAC-A2, etc.) is designed to contain SE elements that specially binds to Analyte A, and the TA elements that specially interacts with the SE and TA elements within the first series of SETAC. Similarly, a second series of SETAC (SETAC-B1, SETAC-B2, etc.) is designed to contain SE elements that specially binds to Analyte B, and the TA elements that specially interacts with the SE and TA elements within the second series of SETAC. Under the designed assay conditions, none of the SE or TA element within the first series of SETAC interact with the SE or TA elements within the second series of SETAC, and vice versa. Distinct signal may be detected for analyte A and analyte B using a scheme illustrated by FIG. 11B, where a first set of secondary amplifiers (SA) and labeling molecules or particles bind specially to the first series of SETAC (SETAC-A1, SETAC-A2, etc.) and a second set of SA and labeling molecules or particles bind specifically to the second series of SETAC (SETAC-B1, SETAC-B2, etc.). Distinct signals are generated by the first and second set of labeling molecules or particles through direct or indirect method, as illustrated in FIG. 5, FIG. 6, and FIG. 8. Similar strategies may be used for detecting more than two analytes simultaneously.

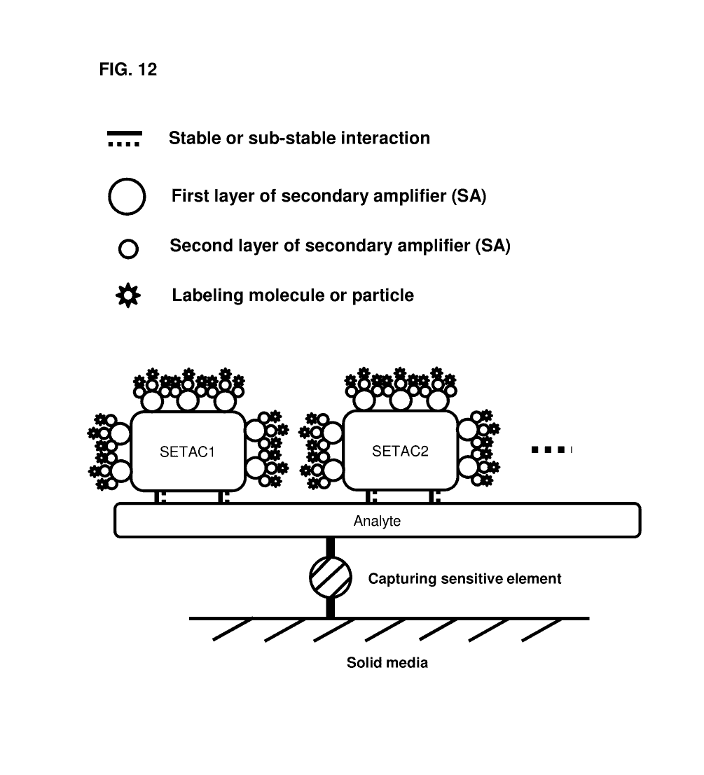

FIG. 12 illustrates a schematic diagram for implementing the biosensor system in one or more embodiments that comprises capturing the analyte to a solid media, such as a glass slide, a plastic slide, a membrane, a bead, a resin, a test strip, a test tube, a single-well plate, and a multi-well plate, followed by detection using the method described in FIG. 1-11

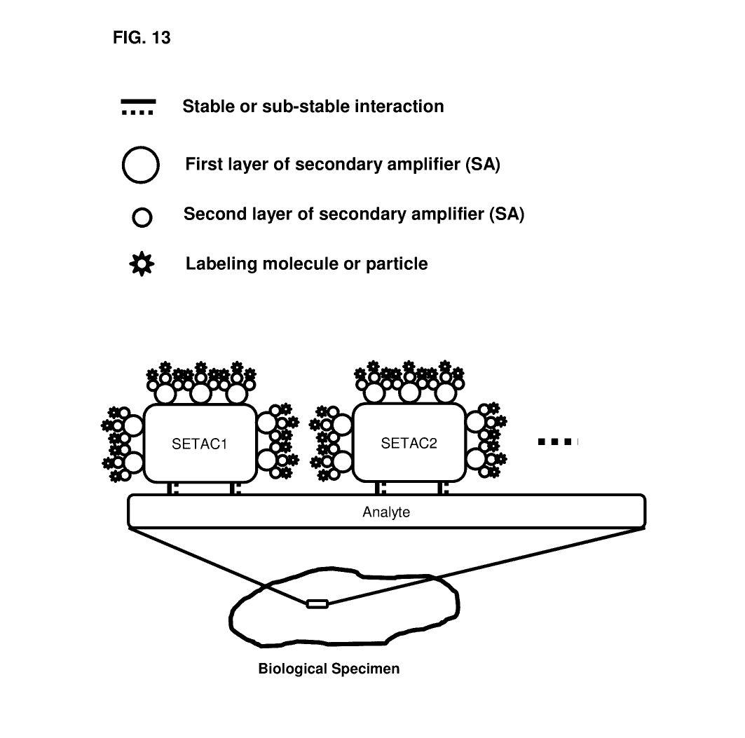

FIG. 13 illustrates a schematic diagram for implementing the biosensor system in one or more embodiments that the analyte may be detected in a biological specimen, such as a cell, a tissue, a fresh-frozen tissue, a fixed tissue, a fixed and embedded tissue, such as formalin fixed and paraffin embedded (FFPE) tissue, blood, plasma, serum, urine, saliva, sputum or any other body fluid, followed by detection using the method described in FIG. 1-11.

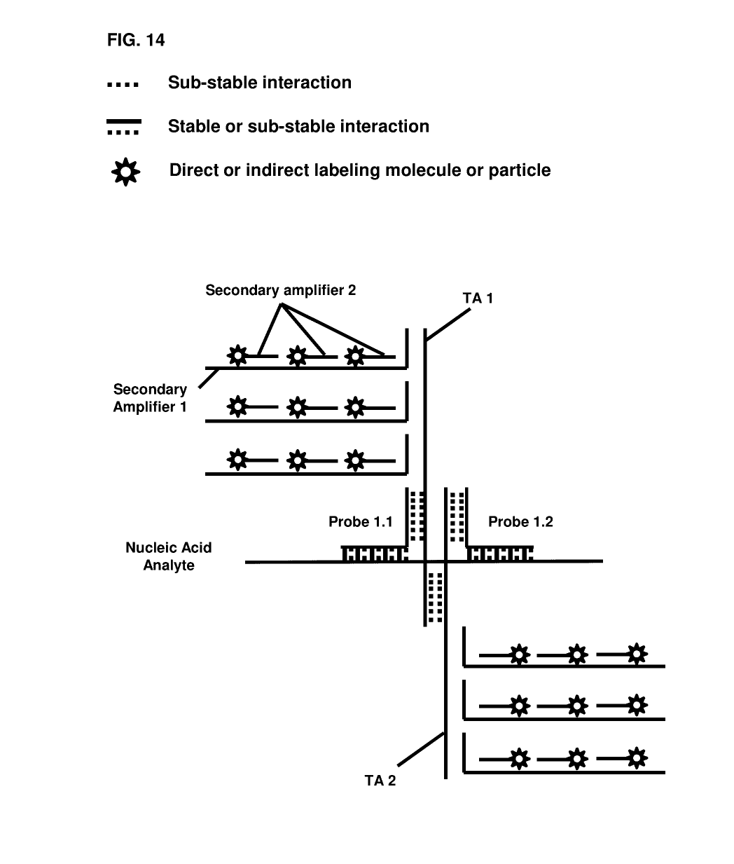

FIG. 14 illustrates a schematic diagram for implementing the biosensor system in one or more embodiments for detecting nucleic acid target, in which it comprises two or more SE (or probes) interacting with the nucleic acid target, two or more TA, one or more additional layers of SA, and direct or indirect labeling molecules or particles. In one or more embodiments, two SE (probes, Probe 1.1 and Probe1.2), interact with the same nucleic acid target through stable or sub-stable interactions, and two signal transducer/amplifiers, TA1 and TA2, interact with Probe 1.1 and Probe 1.2, respectively, through sub-stable interaction. TA1 and TA2 also interact with each other through sub-stable interaction. Together, TA1 and TA2 are bound to the target through cooperative interaction with Probe1.1 and Probe1.2 and between each other.

Similar to what is illustrated in FIG. 6, multiple layers of secondary amplifiers (SA) may be added to enhance the signal. The first layer of the one or more secondary amplifiers, SA1, may be added and bind to TA1 and TA2. Each TA may contain multiple binding sites for SA1. Another layer of secondary amplifier, SA2, may be added and bound to SA1, and each SA1 may have multiple binding sites for SA2. More layers of amplification may be added in the similar manner.

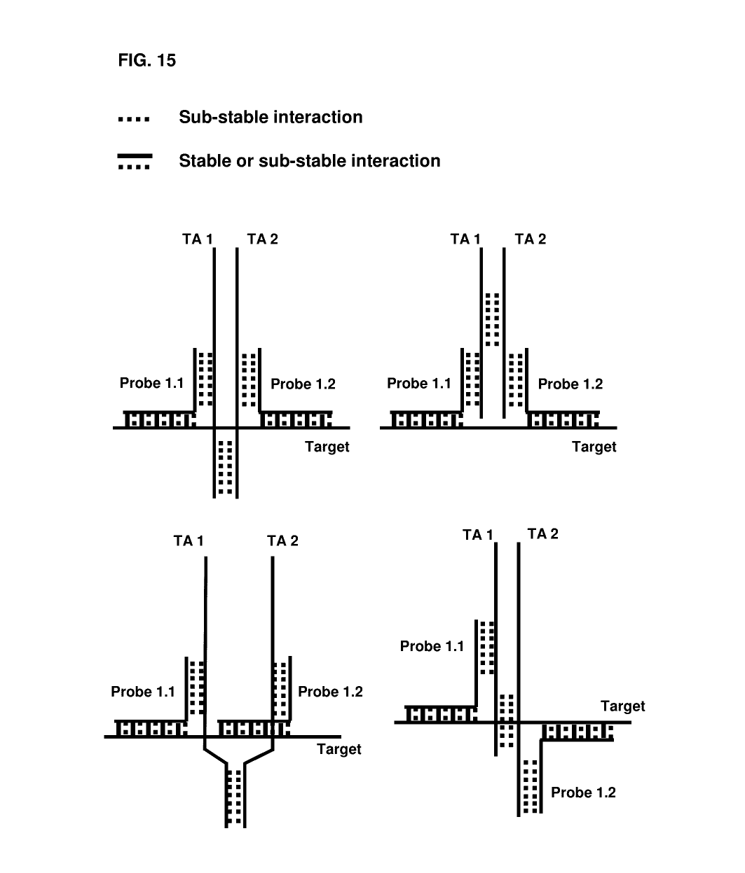

FIG. 15 illustrates a schematic diagram for implementing the biosensor system in one or more embodiments for detecting nucleic acid target, in which the orientations and configurations of the SE/probes and the TA may be different, as long as the SE/probes and TA are held together through multiple cooperative interactions to form a stable SETAC.

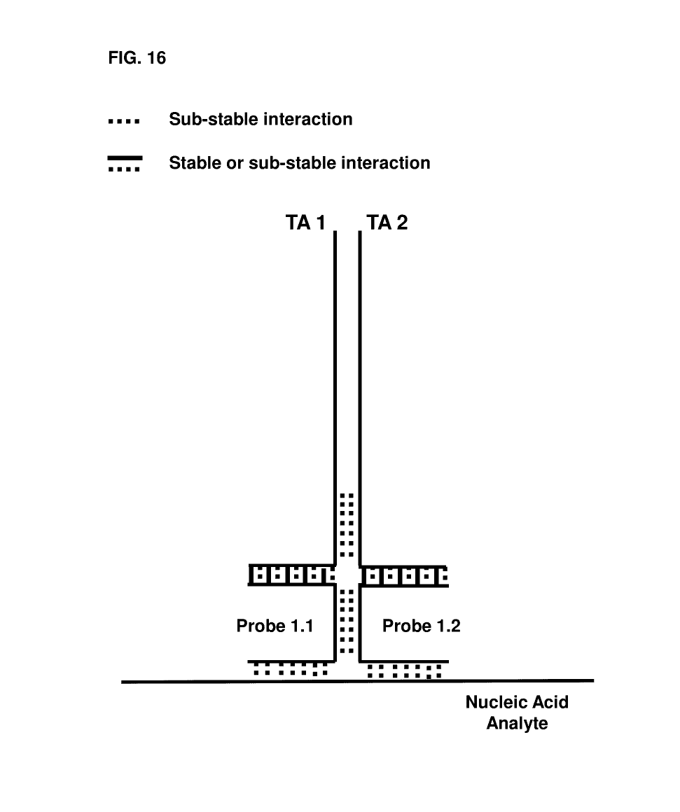

FIG. 16 illustrates a schematic diagram for implementing the biosensor system in one or more embodiments for detecting a nucleic acid, where all the interactions among the components in an SETAC are sub-stable. In one or more embodiments, under a designed assay condition, the pair of SE/probes binds to the analyte through a sub-stable interaction. A TA interact with a SE/probe or another TA, and a SE/probe interacts with another SE/probe through sub-stable interactions. The collective probe-probe, probe-TA, TA-TA sub-stable interaction forms a stable SETAC complex.

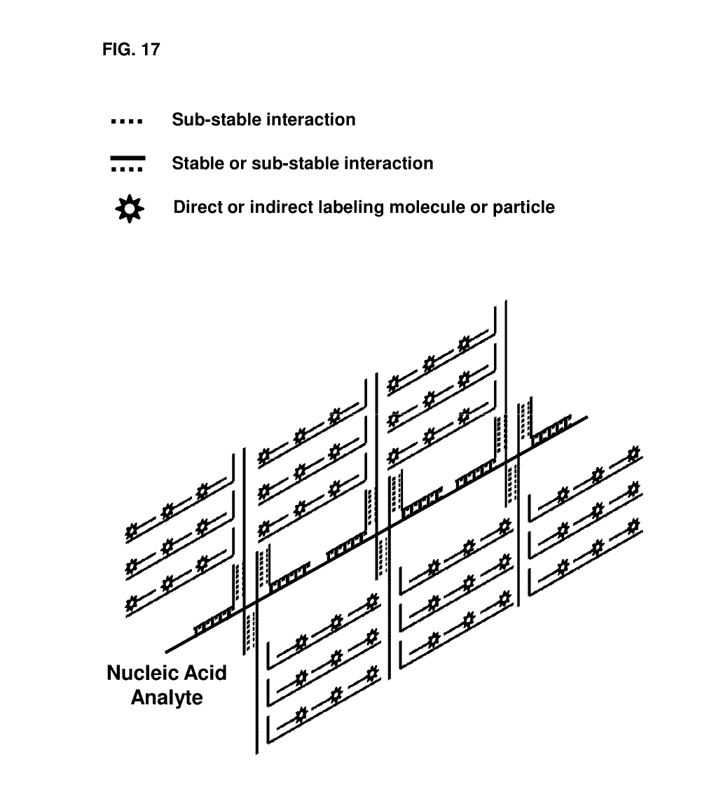

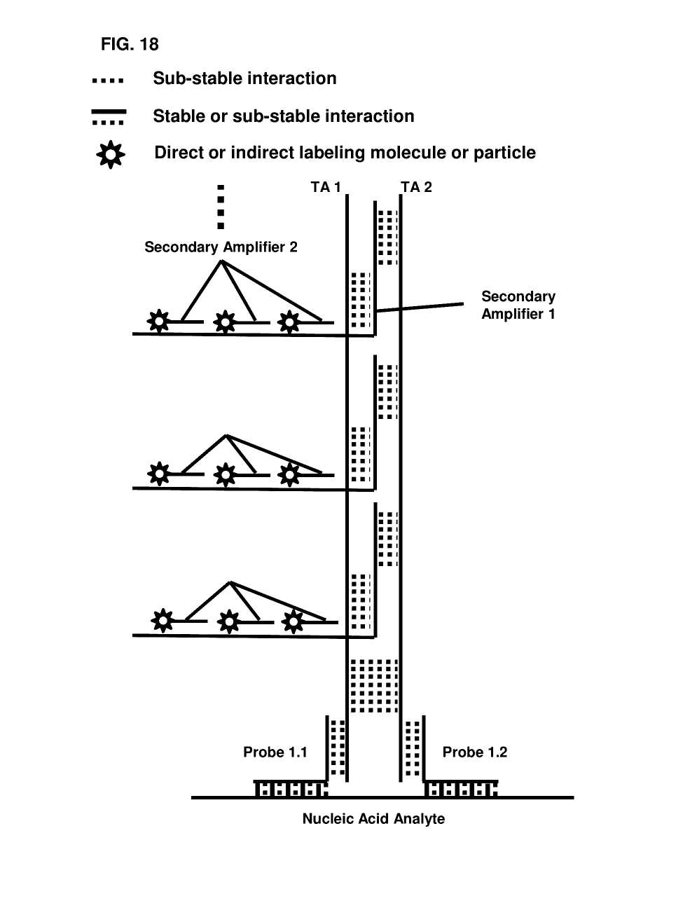

FIG. 17 illustrates a schematic diagram for implementing the biosensor system in one or more embodiments for detecting a nucleic acid that comprises two or more SE/probe-TA complexes, as illustrated in FIG. 9 and FIG. 10, which comprises different SE/probes, but same set of TA. Multiple layers of secondary amplifiers may be added to enhance the signal. TA may have multiple binding sites for the first layer of SA, and each layer of the SA may contain multiple binding sites for the next higher layer of SA, as illustrated in FIG. 14. FIG. 18 illustrates a schematic diagram for implementing the biosensor system in one or more embodiments, in which one or more layers of secondary amplifier (SA) interact with TA, as illustrated in FIG. 7, through sub-stable interaction for detecting nucleic acid analytes.

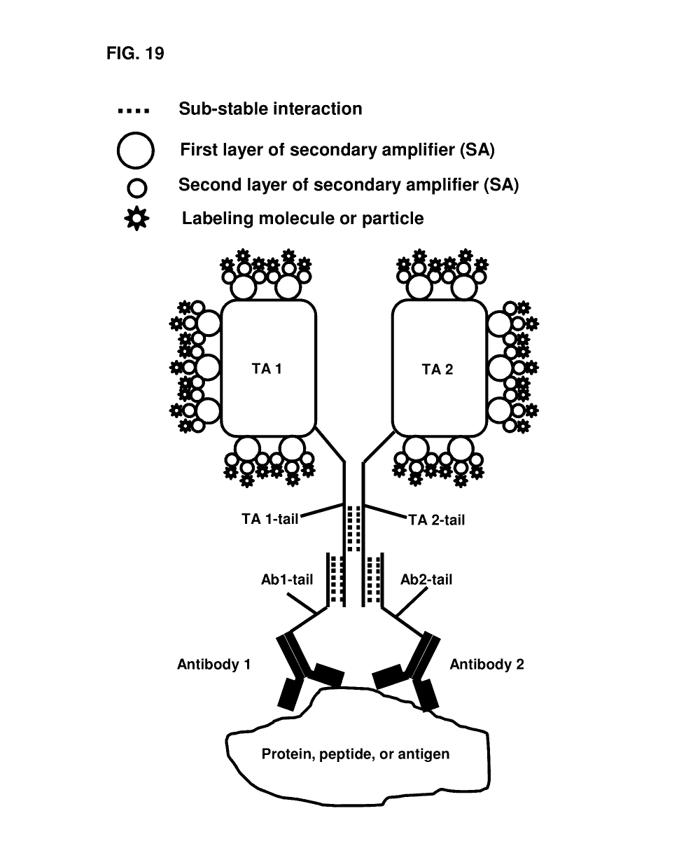

In one or more embodiments, the analyte may be a protein or fraction of a protein, a peptide or fraction of a peptide, or an antigen, as illustrated in FIG. 19. In designed assay conditions, each SE comprised an antibody (or fraction of an antibody) potion which binds to the analyte, conjugated to a nucleic acid tail, which interacts with a TA through sub-stable interaction. Similar to what is illustrated in FIG. 6, multiple layers of secondary amplifiers may be added to enhance the signal.

Multiplex Format.

In some embodiments, as illustrated in FIG. 11, the multiplex format of the biosensor system may be utilized for the simultaneous detection of multiple analytes of any given class comprising DNA, RNA, protein, peptide, or other antigen, under a designed assay condition. The designed assay condition may be controlled such as by the incubation buffer formulation, the incubation temperature, the incubation time, the wash buffer formulation, the washing temperature, and the washing time, to allow different SETAC, optional secondary amplifiers, as well as labeling molecules or particles to associate specifically with the respective analytes, while not interfere or cross-react with each other.

Automated Format.

In some embodiments, the biosensor system may be integrated or it may be transferred to an automated equipment (e.g., an automated slide-stainer). The biosensor system may be implemented for the detection of analyte in a biological sample and be accessible by various automated equipment such as various automated staining equipment or automated stainer. The automated equipment may access biosensor assay procedures of any biosensor format described in FIG. 1-22 as an input in some embodiments, or the biosensor procedures may further comprise machines with accessible codes to drive the automated equipment to perform the biosensor assay according to the procedural sequence set forth in the assay procedure.

In some embodiments, a biosensor procedure may be performed in a specific format as required by particular automated staining equipment. In some of these embodiments, the biosensor procedure may be generated according to a template specifically designated for the particular automated staining equipment. Some embodiments further invoke a translator, a compiler, a mapping, or a transformer, etc. to translate, compile, map, or transform a biosensor procedure from one format to another format.

A biosensor assay format described herein may be transferred to automated staining equipment in various different ways. In some embodiments, a biosensor assay procedure may be transmitted to automated staining equipment via one or more network components such as the Internet, an intranet, or a wide-area network. In some other embodiments, a biosensor procedure may be stored in a persistent storage (e.g., flash memory, an optical disk, an external storage device, etc.) that may be accessed by automated staining equipment.

Advantages.

In some embodiments, under a preferred assay condition, the biosensor system provides an increased sensitivity and specificity through multiple cooperative interactions between two or more SE and at least two TA. Because some of the interactions between SE-analyte, SE-SE, SE-TA, and TA-TA, are sub-stable, only when all the components are present with a proper arrangement as illustrated in any of FIG. 1-20, do they form a stable SETAC complex. The main advantages of this system include:

1) This biosensor system enhances detection sensitivity. One stable SETAC contains multiple TAs, as illustrated in any of FIG. 1-20. Each TA has binding site for optional secondary amplifier or labeling molecules or particles to amplify signal. The signal is amplified by N fold when the TA set for a SETAC contains N TAs (N2) For example, if an SETAC contains two TA, the signal ratio of SETAC and single TA is 2:1; and if the SETAC contains three TA, the ratio is 3:1, so on and so forth.

2) This biosensor system enhances detection specificity. When one of the SE binds non-specifically to another target, it may not generate signal through the TA because one SE alone cannot sustain a stable SETAC structure. Similarly, when one of the TA binds non-specifically to another target, although it may generate background signal, the level of signal is substantially lower than that from a full SETAC, as explained in the first advantage. This is a significant improvement over a traditional amplifier, which generates signal that is equivalent to the signal generated by one sensitive element and adversely affects the limit of detection (LOD) and limit of quantification (LOQ). A further implementation as illustrated in FIG. 7 and FIG. 18 further reduces background because the secondary amplifier only binds to two or more TA through cooperative sub-stable interaction. Therefore, a single non-specifically bound TA will not generate background signal.

3) The biosensor system synergistically enhances the S/N ratio of the detection system. The numerator, i.e. signal, is increased by N fold, where N equals the number of TA in each SETAC complex, and the denominator, noise, i.e. background is decreased by increased specificity. The signal ratio of SETAC versus a single TA may be controlled by the total number of TA present in the SETAC. For example, if an SETAC contains two TAs, the signal ratio of SETAC and single TA is 2:1; and if the SETAC contains three TAs, the ratio is 3:1. Therefore, the S/N ratio of the biosensor system may be controlled by the design of the SETAC for the best benefit of the overall assay performance.

4) The biosensor system is highly scalable. An assay for detecting a new analyte may be developed very fast, because the TA may be designed to interact with the SE through a common mechanism. For example, both the SE the TA may contain a specific nucleic acid tail that interact with each other through hybridization, as illustrated in FIG. 14-19. In this way, to develop an assay to detect a new analyte, it only requires finding at least two SE that specifically recognize different regions of the analyte, conjugating the nucleic acid tail to the SE, and then the identical TA system may be utilized. If the analyte is a nucleic acid, such as DNA or RNA, then the SE/probes may be designed through a computer algorithm to select probe sequences that specifically bind to the target under the designed conditions, such as by calculating the Tm of the probe and based the homology of the probe to potential non-specific targets (as the whole genome of many species are publicly available). The probes may then be synthesized chemically with fast turnaround time.

5) This biosensor system allows multiplex detection of analyte using minimum number of distinct signal types. Two or more independent SETAC may be designed in a way that neither the SE nor the TA in one SETAC will interact with the SE or TA in the other SETAC. As a result, these SETAC may independently and simultaneously detect multiple analytes. Moreover, if different TA in one SETAC produce different signal types, such as different fluorescent signals, the combined signal that the SETAC generates may be distinct from any of the signal generated from a single TA. For example, in an SETAC that contains 2 TA, if both TA produce signal type A, the combined signal from the SETAC will be type A, if both TA produce signal type B, then the combined signal from the SETAC will be type B. However, if one TA produces signal type A and the other TA produces signal type B, the combined signal from the SETAC will be type A+B, which is different from both type A and signal type B. As a result, with only two distinct signal types (A and B), a total of 3 distinct signal patterns may be produced (A, B, and A+B), which allows multiplex detection of 3 analytes. Similarly in a SETAC that contains total number of n TA, with total number of m signal types, the total number of distinct signal patterns generated from the combination of different numbers of signal types will be much larger than m, which allows much higher plex detection from only a small number of signal types. This system may be used in combination with different signal categories, such as fluorescent molecules, barcode labels, isotopic elements, etc.

Biosensor System.

Various embodiments are related to a biosensor system that may be used to detect nucleic acids, proteins, peptides, or other antigens present in a biological sample. Such a system contains at least 2 sensitive elements (SE). For detecting a nucleic acid, the SE may be a natural nucleic acid fragment, a oligonucleotide, which comprises deoxynucleotide, ribonucleotide, nucleotide derivatives, and a mix of two or more of those types. For detecting a protein, a peptide, or an antigen, the SE may contain a part of a monoclonal antibody, a fragment of monoclonal antibody, a polyclonal antibody, a fragment of polyclonal antibody. For detecting an antibody, the SE may contain a peptide, a fragment of a peptide, a fragment of a naturally occurring antigen, or a hapten. The SE may also contain a part that is different from the analyte binding part, such an oligonucleotide, this part may be used to interact with TA or other SE.

In addition to the sensitive elements, the biosensor system also contains two or more primary signal transducer/amplifier (TA). Each TA specifically binds to a sensitive element and or another TA. When all the SE and the TA are present, a stable sensitive element-transducer/amplifier complex (SETAC) forms under a designed condition. The interaction between any interacting components within the SETAC is sub-stable under the designed assay conditions, such that the absence of one component of the SETAC will result in the SETAC to be sub-stable during incubation or subsequent wash steps. The benefit of such design is that if an SE binds non-specifically to an off-target, it will not sustain a stable SETAC without all the other SE present and interacting with it. Because the chance that all the SE bind to the same non-specific substance within close proximity is substantially low, this design will result in further improved signal-to-noise ratio comparing to using a single SE to recognize the analyte. Furthermore, because the detectable signal is generated through TA, Within an SETAC, each TA is capable of generating signal, and the collective signal generated by the SETAC is the sum of the signal generated by all the composing TA. Therefore, when at least two TA are present in the SETAC, the collective SETAC signal is at least 2 times as high as the signal generated by a single TA. In addition, given that the interaction between any two TA is sub-stable under the designed incubation and washing conditions, the chance that a non-specifically bound TA attracts other TA is substantially low thus background signal from nonspecifically bound TA is low. Therefore, the signal generated by an SETAC, which specifically binds to the analyte, is substantially higher than the background signal from any single non-specifically bound TA, leading to further improved signal-to-noise ratio.

The biosensor system may optionally contain multiple layers of secondary signal amplifiers. The presence of multiple layers of the secondary signal amplifier molecules allows the detection of the analyte at a trace amount scale.

In addition, the biosensor system also contains a labeling system or a detection system, which comprises a labeling molecule or particle that directly or indirectly generates detectable and quantifiable signal. In some embodiments, the labeling molecules may be a nucleic acid, an oligonucleotide, a deoxynucleotide, a ribonucleotide, a nucleotide derivative, a monoclonal antibody, a fragment of monoclonal antibody, a polyclonal antibody, a fragment of polyclonal antibody, a protein, a fragment of protein, a peptide, a fragment of peptide, an antigen, a hapten, a biotin, an avidin, a streptavidin, a tyramide signal amplification system, an enzymes, a particle or a mixture of any listed above.

Biosensor Assay Kit.

Various embodiments are related to a biosensor assay kit that may be used to perform the biosensor assay method described in one or more embodiments. The biosensor assay kit contains at least two or more sensitive elements (SE) that specifically bind to the analyte of interest in a biological sample. The biosensor assay kit further contains at least two signal transducer/amplifier (TA), in which the TA and the SE form stable SETAC. In some embodiments, the biosensor assay kit also contains secondary amplifier molecules. The biosensor assay kit further contains a labeling molecule or particle that is for signal detection and quantification. It further contains one or multiple kinds of incubation buffer, or optionally one or multiple kinds of wash buffer.

The label molecule may be a direct labeling system that generates detectable and quantifiable signals by the molecule itself. Example of a direct labeling is a fluorescent label, a colormetric label, a radioactive label, an isotopic label, or a barcode. The labeling molecule or particle may be an indirect labeling system that generates detectable and quantifiable signals through an additional reaction. Examples of an indirect labeling is a horse radish peroxidase (HRP) with substrates such as 3,3′-Diaminobenzidine (DAB), 2,2′-azino-bis(3-ethylbenzothiazoline-6-sulphonic acid) (ABTS), 3-Amino-9-ethylcarbazole (AEC), 3,3′,5,5′-Tetramethylbenzidine (TMB), o-Phenylenediamine dihydrochloride (OPD), AmplexRed, Homovanillic acid, Luminol; alkaline phosphatase (AP) with substrates such as BCIP/NBT, FastRed, FastBlue, 4-Methylumbelliferyl phosphate (4-MUP), p-Nitrophenyl Phosphate; luciferase with substrates such as luciferin, and tyramide signal amplification system.

### Example 1

### Detection of the Presence of HPV 16 and HPV 18 Nucleic Acid with In-Situ Hybridization

Human Papillomaviruses (HPVs) are a group of viruses that contain more than 200 different kinds. About 40 types spread through sexual contact from skin and mucous membranes. It has been demonstrated to be the main causes for various types of cancers including cervical, oral, anal, and head and neck. Sexually transmitted HPV types fall in to two major categories: low risk HPVs and high risk HPVs. High risk HPV types may cause cancer. There are 14 high-risk HPV types that have been identified and studied. These 14 high-risk types are types 16, 18, 31, 33, 35, 39, 45, 51, 52, 56, 58, 59, 66 and 68. Two of these, type 16 and type 18, have been proved to be responsible for approximately 70% of the HPV-caused cancers.

As an example of the biosensor methods in some embodiments, A model system was designed to detect HPV transcripts in formalin fixed and paraffin embedded (FFPE) cell pellets. Two sets of sensitive elements were designed to target HPV16 E6/E7 and HPV18 E6/E7 transcripts, respectively. Each sensitive element set contains a series of paired target probes/SE. The first of each paired probes, SE(N.1), interacted with a TA (TA1) through a nucleotide sequence that is 11-17 nt in length, and the second of each paired probes, SE(N.2), interacted with a different TA (TA2) through a different nucleotide sequence that is 11-17 nt in length. TA1 and TA2 interacted with another through a nucleotide sequence that is 11-17 nt in length and is different from the sequences through which TA1 and TA2 interacted with SE (N.1) and SE(N.2) respectively. Each of the probes were designed to target the HPV transcripts at a region that is 14-60 nucleotide (nt) in length, with a Tm between 40° C.-85° C. at 1M Na+ concentration and have <95% homology to any human transcripts.

A hybridization buffer was designed to create a stringency-dependent environment such that the binding between the SE and TA, and between TA1 and TA2 are sub-stable. Therefore, the desired binding between SE(N.1) and TA1, SE(N.2) and TA2 are sub-stable, the other undesired binding and non-specific binding are also not stable. When the sensitive elements SE(N.1) and SE(N.2), and the TA1 and TA2 formed quadru/tetra-structure, the dynamic sub-stable interactions among SE(N.1), SE(N.2), TA1 and TA2 held the structure in a stable form and built a foundation for subsequence signal amplification and/or detection. The subsequent washing step removed all the other undesired and non-specific species as the interaction other than the quadru/tetra SETAC are all sub-stable interactions only.

Further, a secondary layer of oligonucleotide amplifier (secondary amplifier 1, SA1) was applied to bind to both TA1 and TA2 through a nucleotide sequence between 15-30 nt in length. TA1 and TA2 both bond to multiple SA1. In the next step, another layer of oligonucleotide amplifier (secondary amplifier 2, SA2) was added and bound to SA1 through a nucleotide sequence that is 15-30 nt in length. Each SA1 binds to multiple SA2. Each SA2 has a horseradish peroxidase (HRP) conjugated to its 5′ end (SA2-HRP). Next, SA2-HRP reacts with tyramide conjugated with biotin (TM-biotin) and precipitation formed around the reaction center. Next, streptavidin conjugated with HRP (SA-HRP) was added and bound to the biotin moiety of precipitated TM-biotin. In the last step, 3,3′-Diaminobenzidine (DAB) substrate is added and reacts with SA-HRP to generate brown colored precipitants and allows visualization of signal from each transcript.

Three cell lines were used to assess the specificity and sensitivity of the probes. 1) CASKI cells, a cervical cancer cell line known to be infected by HPV16; 2) HELA cells, a cervical cancer cell line known to be infected by HPV18; and 3) C33A, a cervical cancer cell line known to be negative of any HPV infection.

FFPE cell pellet slides were deparaffinized with Xylene and washed with 100% ethanol. The slides were then treated with epitope retrieval buffer (10 mM Citrate) at 100° C. for 15 min followed by protease treatment (protease K at 1 μg/ml) for 30 min at 40° C. The slides were washed with dH2O and subjected to SE hybridization with SE(N.1) and SE(N.2) in SE hybridization buffer (1-10×SSC, 10-50% formamide, <1% Tween 20, and <1% blocking reagent) for 2 hours at 40° C. followed by washing with wash buffer (0.01-0.5×SSC, 0.01-0.5% SDS) 2 times at room temperature.

The slides were then incubated with primary amplifier set (TA1 and TA2) in amplifier hybridization buffer 1 (1-10×SSC, 10-40% formamide, <1% Tween 20, and <1% blocking reagent) for 30 min at 40° C. followed by washing with wash buffer 2 times at room temperature. The slides were then incubated with the secondary amplifier 1 (SA1) in amplifier hybridization buffer 1 for 30 min at 40° C. followed by washing with wash buffer 2 times at room temperature. The slides were then incubated with the secondary amplifier 2 (SA2) in amplifier hybridization buffer 2 (1-5×SSC, <1% blocking reagent) for 15 min at 40° C. followed by washing with wash buffer 2 times at room temperature.

The slides were then incubated with tyramide amplification buffer (PerkinElmer) prepared according to the manufacturer's recommendation for 20 min at room temperature followed by washing with wash buffer 2 times at room temperature. The slides were then incubated with streptavidin-HRP conjugate prepared according to the manufacturer's recommendation for 20 min at room temperature followed by washing with wash buffer 2 times at room temperature. The slides were then incubated with DAB substrate for 5 min at room temperature followed by washing with dH2O. Counter staining by hematoxylin stain (Gill's hematoxylin) was then performed.

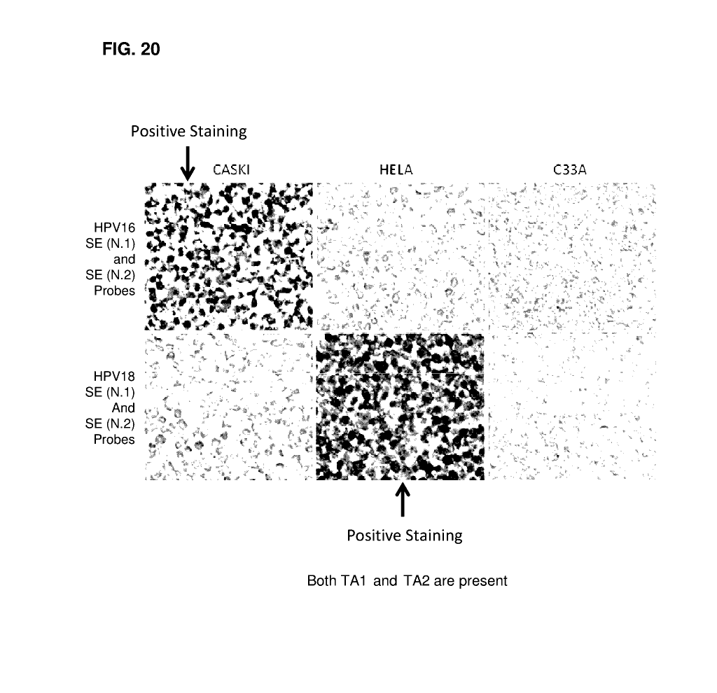

FIG. 20 illustrates cell images obtained after the procedures described above. CASKI, HELA and C33A cells were used to represent the biosensor system in one or more embodiments, in which two sensitive element sets, HPV 16 and HPV 18 SE/probes, and both signal amplifying probes (TA1 and TA2) were used.

As shown in FIG. 20, the HPV16 probe set only detected signal in CASKI cells but not in HELA or C33A cells, whereas HPV18 probe set only detected signal in HELA cells but not in CASKI or C33A cells, demonstrating the specific detection of the sensitive element sets to each HPV subtype.

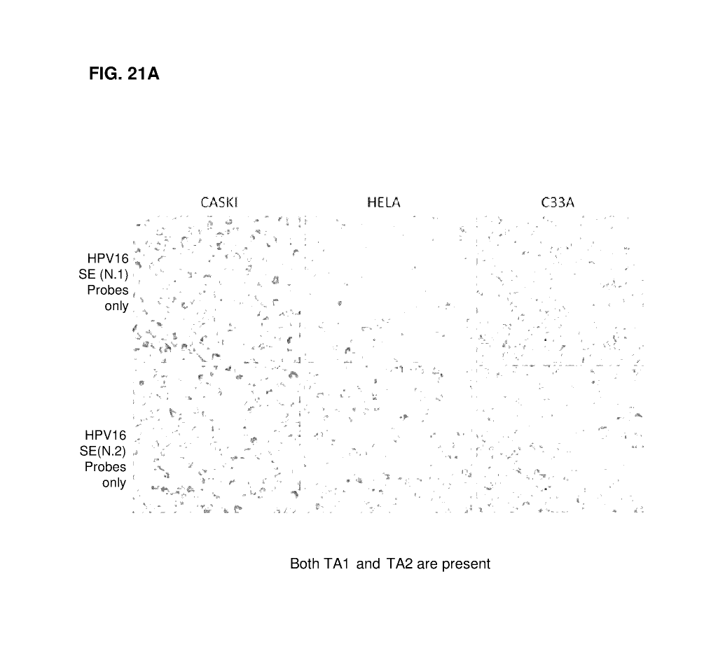

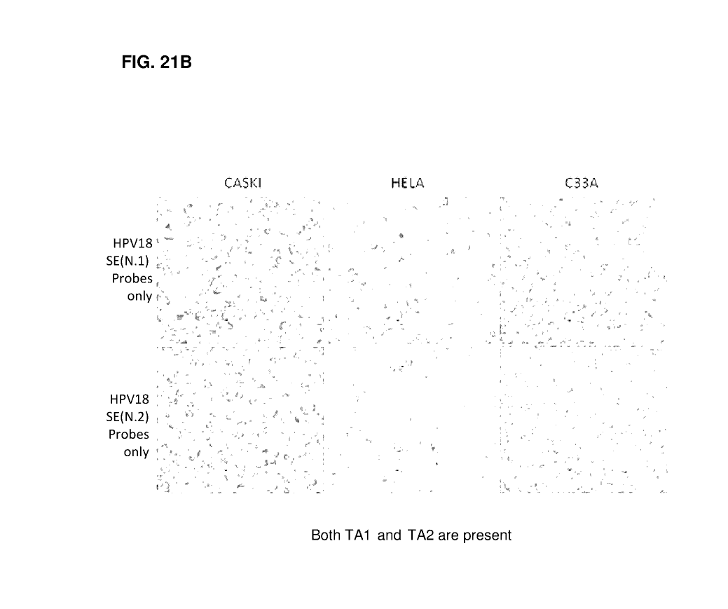

To demonstrate that the detection is dependent on the cooperative hybridization between SE/probes and TA, only half set of each SE/probe pairs (SE(N.1) only or SE(N.2) only) were mixed and used to detect HPV transcripts in these cell lines. FIG. 21A illustrates in-situ hybridization images using CASKI, HELA and C33A cells to represent the biosensor system, in which only half of the HPV 16 SE/probe set was used. FIG. 21B illustrates in-situ hybridization images using CASKI, HELA and C33A cells to represent the biosensor system, in which only half of the HPV18 SE/probe set was used.

As shown in FIG. 21, neither SE(N.1) probe mix alone nor SE(N.2) probe mix alone detected any signal in any of the three cell lines, demonstrating that the detection requires a pair of SE/target probes to form a cooperative hybridization structure to allow signal amplification.

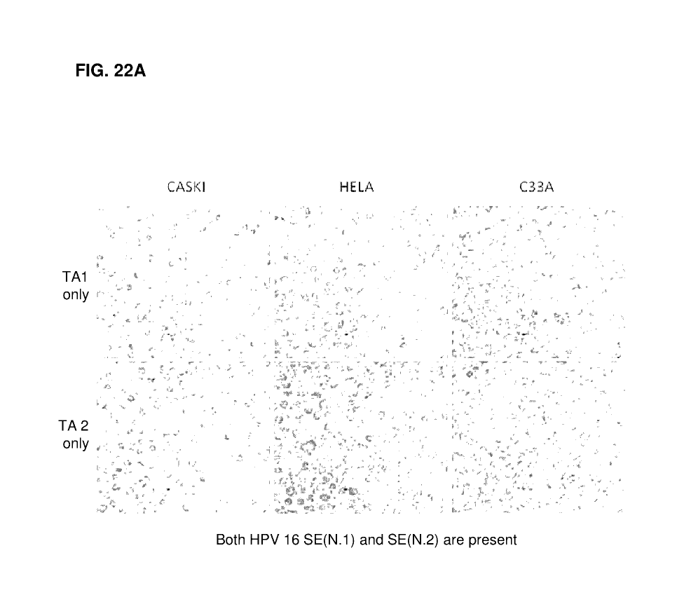

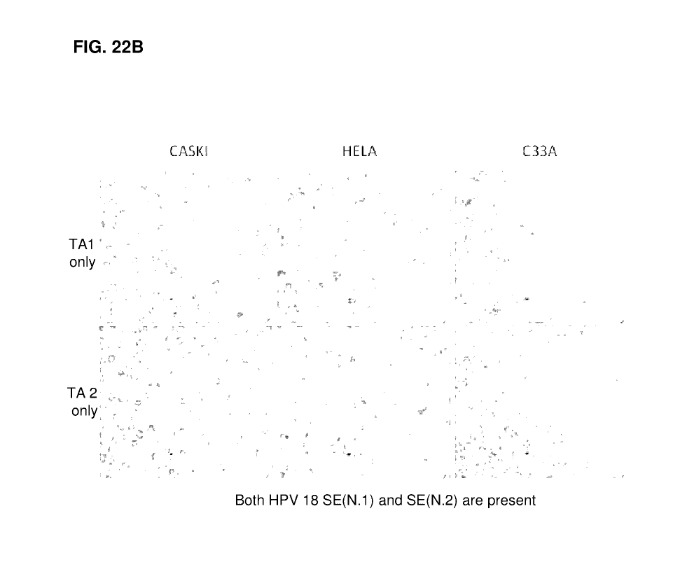

To demonstrate that both signal transducing/amplifying probes (TA1 and TA2) are required for generating signal, only TA1 or only TA2 was added after hybridizing with the full set of HPV16 or HPV18 probes consisting of both SE(N.1) and SE(N.2). FIG. 22 illustrates in-situ hybridization images using CASKI, HELA and C33A cells to represent the biosensor systems, in which the full HPV16 or HPV18 SE/probe set and only one of the two TA was used.

FIG. 22A illustrates in-situ hybridization images using CASKI, HELA and C33A cells to represent the biosensor system, in which the full HPV 16 SE/probe set and only one of the two amplifiers was used; and FIG. 22B illustrates in-situ hybridization images using CASKI, HELA and C33A cells to represent the biosensor system, in which the full HPV 18 SE/probe set and only one of the two TA was used.

As shown in FIG. 22, neither TA1 nor TA2 alone enabled detection of HPV16 (FIG. 22A) or HPV18 (FIG. 22B) despite the presence of the entire SE/probe sets.

The above results together demonstrated that only when all the components (SE(N.1), SE(N.2), TA1, and TA2) are present, a stable quandro/tetra structure (SETAC), which is required for detecting specific signal from the targets, may be formed.

The method used for detecting the HPV 16 and HPV18 nucleic acid targets, as described above, can be used for detecting any nucleic acid targets in a biological sample. At least two probes can be designed to bind to a nucleic acid target, such as DNA or RNA. The probes bind to the nucleic acid target through a target binding region in each probe. The target binding region of at least one of the probes is designed to have <95% homology with any other nucleic acid in the biological sample, such that under the designed conditions, the probe only binds to the nucleic acid target of interest, but not other nucleic acids. As a result, the probe/TA complex can only bind specifically to the nucleic acid target of interest, but not other nucleic acids in the biological sample, under the designed conditions. Each of the two or more probes also contains a signal transducer/amplifier (TA) interacting region that interact with the TA. The TA interacting regions of all the probes can be identical to those of the probes used for detecting HPV16 and HPV18 nucleic acid, as described in the example above, but they can also be designed differently but function in a similar way as the example demonstrated.

We claim:

1. A method for detecting an analyte in a biological sample, comprising the steps of:
obtaining a biological sample that contains an analyte of interest;
interacting the analyte with two or more sensitive elements (SE), wherein
each of the SE specifically recognizes, interacts, and binds to a region of the analyte of interest under a first designed assay condition, wherein
the first designed assay condition results in a first stringency-dependent interaction between the analyte and the sensitive elements;
interacting the SE with two or more primary signal transducing/amplifing elements (TA), wherein
each of the TA specifically recognizes, interacts and binds to one or more of the SE, and/or one or more of the TA under a second designed assay condition, wherein
the second designed assay condition results in a second stringency-dependent interaction, and
the two or more SE and the two or more TA collectively form a substantially stable SE/TA complex (SETAC) that binds to the analyte.
generating a signal through a labeling system that comprises a labeling molecule or a labeling particle; and
detecting and quantifying the signal, wherein the signal is in proportion to a quantity of the analyte present in the biological sample.

2. The method of claim 1, wherein
the second stringency-dependent interaction is sub-stable, and
at least one interaction between the TA and another TA is sub-stable.

3. The method of claim 1, wherein
the second stringency-dependent interaction is a sub-stable interaction,
all the interactions between the SE and the TA are sub-stable, and
all the interactions between one of the TA and another TA are sub-stable.

4. The method of claim 1, further comprises immobilizing the analyte or the biological sample on a solid media selected from the group consisting of a glass slide, a plastic slide, a membrane, a bead, a resin, a test strip, a test tube, a single-well plate, and a multi-well plate.

5. The method of claim 1, wherein
the analyte is selected from a group comprising a nucleic acid, an antibody and an antigen, wherein
the nucleic acid is selected from the group consisting of DNA, messenger RNA (mRNA), and non-messenger RNA that is selected from the group consisting of transfer RNAs (tRNA), ribosomal RNA (rRNA), snoRNA, microRNA, siRNA, snRNA, exRNA, piRNA and scaRNA;
the biological sample is selected from the group consisting of a cell, tissue, blood, serum and body fluid; or
the sensitive element is selected from the group comprising a nucleic acid, an oligonucleotide, a deoxynucleotide, a ribonucleotide, a nucleotide derivatives, a monoclonal antibody, a fragment of monoclonal antibody, a polyclonal antibody, a fragment of polyclonal antibody, a protein, a fragment of protein, a peptide, a fragment of peptide, an antigen, a hapten, a particle and the combination thereof.

6. The method of claim 1, wherein
the labeling system is a direct labeling system or an indirect labeling system, and
the labeling system is selected from the group consisting of a fluorescent labeling system, a chemiluminescent labeling system, a radioactive labeling system, an enzymatic labeling system, a nanoparticle labeling system, and a barcode labeling system; or
the labeling molecule or the labeling particle is selected from the group consisting of a nucleic acid, an oligonucleotide, a deoxynucleotide, a ribonucleotide, a nucleotide derivative, a monoclonal antibody, a fragment of monoclonal antibody, a polyclonal antibody, a fragment of polyclonal antibody, a protein, a fragment of protein, a peptide, a fragment of peptide, an antigen, a hapten, a biotin, an avidin, a streptavidin, a tyramide signal amplification system, an enzymes, a particle, and the combination thereof.

7. The method of claim 1, wherein the steps of interacting the two or more SE with the analyte and the steps of interacting the two or more TA with the two or more SE may occur concurrently.

8. The method of claim 5, wherein
the enzymatic label is selected from the group consisting of a horseradish peroxidase (HRP), an alkaline phosphatase (AP), and a luciferase; and
the enzymatic label requires a substrate for signal generation, wherein
the substrate is selected from the group consisting of 3,3′-Diaminobenzidine (DAB), 2,2′-azino-bis(3-ethylbenzothiazoline-6-sulphonic acid) (ABTS), 3-Amino-9-ethylcarbazole (AEC), 3,3′,5,5′-Tetramethylbenzidine (TMB), o-Phenylenediamine dihydrochloride (OPD), AmplexRed, Homovanillic acid, Luninol, BCIP/NBT, FastRed, FastBlue, 4-Methylumbelliferyl phosphate (4-MUP), p-Nitrophenyl Phosphate, and luciferin.

9. The method of claim 1, further comprises interacting the TA with additional one or more layers of a secondary amplifier (SA) wherein
the secondary amplifer (SA) is selected from the group consisting of a nucleic acid, an oligonucleotide, a deoxynucleotide, a ribonucleotide, a nucleotide derivative, a monoclonal antibody, a fragment of monoclonal antibody, a polyclonal antibody, a fragment of polyclonal antibody, a protein, a fragment of protein, a peptide, a fragment of peptide, an antigen, a hapten, a biotin, an avidin, a streptavidin, a tyramide signal amplification system, an enzymes, a particle, and the combination thereof;
the first layer of the SA specifically binds to the TA;
each additional layer of the SA specifically binds to the SA of previous layer; and
the labeling system specifically binds to the TA or the SA of the most outer layer.

10. The method of claim 1, wherein the first or the second stringency dependent interaction results in a stable interaction between two or more interacting molecules.

11. The method of claim 1, wherein the first or the second stringency dependent interaction results in a sub-stable interaction between two or more interacting molecules.

12. The method of claim 1, wherein the first designed assay condition or the second designed assay condition comprises a composition of an incubation solution, an incubation temperature, and an incubation time.

13. A biosensor assay kit for implementing the method of claim 1, comprising:
two or more sensitive elements (SE) that specifically bind to the analyte,
two or more signal transducer/amplifier (TA) that specifically bind to the SE and to at least one of the TA,
one or more secondary amplifiers (SA) that specifically bind to the TA,
a labeling molecule that provides, directly or indirectly, a detectable signal, and
at least one type of incubation buffer.

14. A method for detecting a nucleic acid target in a biological sample, comprising the steps of:
obtaining a biological sample that contains a nucleic acid of interest;
interacting the nucleic acid target with two or more probes, wherein
each of the probe specifically recognizes, interacts, and binds to a region of the nucleic acid target of interest under a first designed assay condition, wherein
the first designed assay condition results in a first stringency-dependent interaction between the probes and the nucleic acid target;
interacting the probes with two or more primary signal transducing/amplifying elements (TA), wherein
each of the TA specifically recognizes, interacts and binds to one or more of the probes, and/or one or more of the TA under a second designed assay condition, wherein
the second designed assay condition results in a second stringency-dependent interaction, and
the two or more probes and the two or more TA collectively form a substantially stable probe/TA complex that binds to the nucleic acid target,
generating a signal through a labeling system that comprises a labeling molecule or a labeling particle; and
detecting and quantifying the signal, wherein the signal is in proportion to the amount of the nucleic acid target present in the biological sample.

15. The method of claim 14, wherein
the second stringency-dependent interaction is sub-stable, and
at least one interaction between the TA and another TA is sub-stable.

16. The method of claim 14, wherein
the second stringency-dependent interaction is a sub-stable interaction,
all the interactions between the probe and the TA are sub-stable, and
all the interactions between one of the TA and another TA are sub-stable.

17. The method of claim 14, further comprises immobilizing the nucleic acid target or the biological sample on a solid media selected from the group consisting of a glass slide, a plastic slide, a membrane, a bead, a resin, a test strip, a test tube, a single-well plate, and a multi-well plate.

18. The method of claim 14, wherein
the nucleic acid target is selected from the group consisting of DNA, messenger RNA (mRNA), and non-messenger RNA that is selected from the group consisting of transfer RNAs (tRNA), ribosomal RNA (rRNA), snoRNA, microRNA, siRNA, snRNA, exRNA, piRNA and scaRNA;
the biological sample is selected from the group consisting of a cell, tissue, blood, serum and body fluid; or
the probe is selected from the group comprising a nucleic acid, an oligonucleotide, a deoxynucleotide, a ribonucleotide, a nucleotide derivatives, and the combination thereof.

19. The method of claim 14, wherein
the labeling system is a direct labeling system or an indirect labeling system;
and is selected from the group consisting of a fluorescent labeling system, a chemiluminescent labeling system, a radioactive labeling system, an enzymatic labeling system, a nanoparticle labeling system, and a barcode labeling system; or
the labeling molecule or the labeling particle is selected from the group consisting of a nucleic acid, an oligonucleotide, a deoxynucleotide, a ribonucleotide, a nucleotide derivative, a monoclonal antibody, a fragment of monoclonal antibody, a polyclonal antibody, a fragment of polyclonal antibody, a protein, a fragment of protein, a peptide, a fragment of peptide, an antigen, a hapten, a biotin, an avidin, a streptavidin, a tyramide signal amplification system, an enzymes, a particle, and the combination thereof.

20. The method of claim 14, wherein the steps of interacting the two or more probes with the nucleic acid target and the step of interacting the two or more TA with the two or more probes occur concurrently.

21. The method of claim 19, wherein
the enzymatic label is selected from the group consisting of a horseradish peroxidase (HRP), an alkaline phosphatase (AP), and a luciferase; and
the enzymatic label requires a substrate for signal generation, wherein
the substrate is selected from the group consisting of 3,3′-Diaminobenzidine (DAB), 2,2′-azino-bis(3-ethylbenzothiazoline-6-sulphonic acid) (ABTS), 3-Amino-9-ethylcarbazole (AEC), 3,3′,5,5′-Tetramethylbenzidine (TMB), o-Phenylenediamine dihydrochloride (OPD), AmplexRed, Homovanillic acid, Luninol, BCIP/NBT, FastRed, FastBlue, 4-Methylumbelliferyl phosphate (4-MUP), p-Nitrophenyl Phosphate, and luciferin.

22. The method of claim 14, further comprises interacting the TA with additional one or more layers of a secondary amplifiers (SA) wherein
the SA is selected from the group consisting of a nucleic acid, an oligonucleotide, a deoxynucleotide, a ribonucleotide, a nucleotide derivative, a monoclonal antibody, a fragment of monoclonal antibody, a polyclonal antibody, a fragment of polyclonal antibody, a protein, a fragment of protein, a peptide, a fragment of peptide, an antigen, a hapten, a biotin, an avidin, a streptavidin, a tyramide signal amplification system, an enzymes, a particle, and the combination thereof.
the first layer of an SA specifically binds to the TA,
each additional layer of the SA specifically binds to the SA of previous layer, and
the labeling system specifically binds to the TA or SA of the most outer layer.

23. The method of claim 14, wherein the first or the second stringency dependent interaction results in a stable interaction between two or more interacting molecules.

24. The method of claim 14, wherein the first or the second stringency dependent interaction results in a sub-stable interaction between two or more interacting molecules.

25. The method of claim 14, wherein the first or the second designed assay condition comprises a composition of incubation solution, an incubation temperature, and an incubation time.

26. A biosensor assay kit for implementing the method of claim 14, comprising:
two or more probes that specifically bind to the nucleic acid target,
two or more primary signal transducer/amplifier (TA) that specifically bind to the probes and to at least one of the TA,
one or more secondary amplifier (SA) that specifically bind to the TA,
a labeling molecule that provides, directly or indirectly, a detectable signal, and
at least one type of incubation buffer.

27. A method for detecting a human papillomavirus (HPV) nucleic acid in a biological sample, comprising the steps of:
obtaining a biological sample;
interacting the biological sample with two or more probes that specifically bind to a HPV E6/E7 transcript or a HPV DNA;
interacting two or more signal transducer/amplifier elements that specifically bind to the probes under a designed hybridization condition, wherein
the designed hybridization condition results in a stringency-dependent interaction between the probe and the signal transducer/amplifier, or between two signal transducers/amplifises; and
the two or more probes and the two or more signal transducer/amplifier collectively form a substantially stable complex; and
interacting the complex with a label molecule; and
detecting and quantifying an amount of the label molecules bound to the complex, wherein
the quantifying the amount of the label molecules comprises quantifying the analyte in proportion to the quantity of the label molecules bound to the complex.

28. The method of claim 28, wherein the HPV E6/E7 transcript or the HPV DNA is selected from the group consisting of HPV type 16, and HPV type 18.
